(19) United States
(12) Patent Application Publication
(10) Pub. No.: US 2020/0246491 A1
(43) Pub. Date: Aug. 6, 2020

(54) HyperPolarising Substrates Through Relayed Transfer Via Systems Containing Exchangeable Protons

(71) Applicant: University of York, York (GB)

(72) Inventors: Wissam Iali, North Yorkshire (GB); Peter Rayner, North Yorkshire (GB); Simon Benedict Duckett, North Yorkshire (GB)

(73) Assignee: University of York, York (GB)

(21) Appl. No.: 16/641,773

(22) PCT Filed: Jul. 24, 2018
(86) PCT No.: PCT/GB2018/052080

§ 371 Date: Feb. 25, 2020

(30) Foreign Application Priority Data
Jul. 25, 2017 (GB) 1711967.8
Sep. 19, 2017 (GB) 1715082.2
Jun. 5, 2018 (GB) 1809221.3

(51) Int. Cl. A61K 49/18; A61B 5/055

(52) U.S. Cl. CPC A61K 49/18; G01R 33/282; A61B 5/055

(57) ABSTRACT

There is described a method for the preparation of a hyperpolarised target molecule, wherein said molecule comprises at least one —OH, —NH or —SH moiety, via proton exchange from a polarisable molecule, said method comprising the steps of:
(i) preparing a fluid containing a transfer catalyst; parahydrogen; and a polarisable molecule containing at least one exchangeable proton, such as, an —OH, —NH or —SH moiety;
(ii) applying a magnetic field or radio frequency excitation such that hyperpolarisation is transferred from parahydrogen to the polarisable molecule when bound to the magnetisation transfer catalyst;
(iii) separately or simultaneously introducing a target molecule, wherein said target molecule contains at least one —OH, —NH or —SH exchangeable proton, enabling hyperpolarisation transfer via proton exchange with the polarisable molecule.

### FIELD OF THE INVENTION

The present invention relates to a method for the production of a hyperpolarised agent via hyperpolarisation transfer.

More particularly, the present invention provides a method for the production of a hyperpolarised agent and the associated signal enhancement of ¹H, ¹³C, ³¹P, ¹⁹F and ¹⁵N responses, in —NH containing species, such as amines and amides, including ammonium species; —OH containing species, such as alcohols, sugars, carboxylic acids, oxalic acids, carbonic acid (including salts of such acids, e.g. salts of oxalic acid, such as Na₂C₂O₄, and salts of carbonic acid, such as NaHCO₃), phosphates, borates, inorganic hydroxides; and their congeners such as —SH species, e.g. thiols, thioamides, etc. Metal hydroxides such as ⁶LiOH, Al(OH)₃, related complexes containing hydroxide or amine ligands, where it extends to ²⁹Si, ⁷⁷Se, ¹¹³Cd, ¹⁹⁹Hg, ¹¹⁷Sn, ¹⁹⁵Pt, ²⁰⁷Pb, ⁵⁷Fe, ⁸⁹Y, ¹⁰⁹Ag and ¹⁸³W. The extension of this effect is achieved by harnessing the exchangeable protons of the —NH, —SH or —OH containing species for hyperpolarisation transfer. The hyperpolarisation transfer described herein is generally referred to as a RELAY effect.

### BACKGROUND OF THE INVENTION

Magnetic resonance imaging (MRI) is a technique based upon the science of nuclear magnetic resonance (NMR). MRI has become particularly attractive to physicians as images of parts of a patient's body thereof can be obtained non-invasively and without exposing the patient and the medical personnel to potentially harmful radiation such as X-rays.

Furthermore, due to its high quality images and good spatial and temporal resolution, MRI is a favourable imaging technique for imaging patients' soft tissue and organs.

One of the main advantages of SABRE is that it achieves this result without the incorporation of p-H₂ into the substrate. This technique is effectively a form of catalysis which utilizes a suitable catalyst^{[4]}, to reversibly bind both H₂ (p-H₂) and the substrate in order to assemble a reaction intermediate in which polarisation is able to transfer, at low magnetic fields, from p-H₂ into the substrate.^{[5]}

NMR and MRI involve the detection of what can be viewed to be transitions of nuclear spins between an excited state and a ground state in an applied magnetic field. Because the energy difference between these states is relatively small, the usual Boltzmann distribution of chemically identical nuclei is such that at room temperature the populations of nuclear spin states which are in dynamic equilibrium are almost identical. Since the strength of the detected signal in magnetic resonance experiments is proportional to the population difference, NMR and MRI signals are typically weak.

The strength of detectable NMR signals can however be enhanced by hyperpolarising the magnetic nuclei. Hyperpolarisation in this context refers to a process in which a significant excess of magnetic nuclei are induced into a spin state. This results in a large increase in available signal due to the much larger inequality of populations across the energy levels that will ultimately be probed. In order for a hyperpolarised state to be useful, it is important that the spin state is sufficiently long lived to provide useful information, i.e. that the relaxation time of the spin state is ‘long’. The rules governing the relaxation rates of nuclear spins are complex but known. It suffices to say that certain nuclei and spins systems have relaxation times which may extend from seconds to hours, days, months or even years.

There are a number of ways to induce certain nuclei into a hyperpolarised state. The simplest way is to cool the material to very low temperatures in the presence of a magnetic field, which will favour population of the lower energy state in which the spins of the nuclei are aligned with the applied magnetic field. This method is suitable for the production of hyperpolarised monatomic gases such as xenon or helium-3. The polarisation levels of these nuclei have also been increased via the use of laser-based technologies.

Hyperpolarisation aims to turn typically weak NMR and MRI responses into strong signals so that normally impractical measurements can be made. The parahydrogen based signal amplification by reversible exchange process (SABRE) has been used previously to hyperpolarise a range of agents that contain multiple bonds to nitrogen. Nuclear magnetic resonance (NMR) reflects one of the most powerful methods to study materials while magnetic resonance imaging (MRI) plays a vital role in clinical diagnosis. However, the low sensitivity of these two techniques acts to limit their applicability, while adding substantially to cost. Remarkably, the hyperpolarisation method, dynamic nuclear polarisation (DNP) has been shown to improve the detectability of agents such as pyruvate so that the MRI based diagnosis of disease through in vivo assessment of metabolism is possible. In contrast, easier to prepare parahydrogen (p-H₂), which exists in a pure nuclear spin state, was shown to enhance the strength of an NMR signal in 1987(1) but progress towards its clinical use has been limited. This reflects the fact it was originally used to detect chemically modified hydrogenation products which acted to limit the range of organic materials it could work with. (2, 3) Only recently, in 2009, was a p-H₂ technique reported where the original chemical identity of the sensitised molecule was retained. (4) This approach is called Signal Amplification By Reversible Exchange (SABRE) and while it has proven to be highly successful for the hyperpolarisation of agents that contain multiple bonds to nitrogen such as nicotinamide (5), isoniazid (6), pyrazole (7), acetonitrile (8), operating on nuclei such as ¹H, ¹³C, ³¹P, ¹⁹F and ¹⁵N (5, 9-13), there are many classes of molecule it fails to sensitise.

SABRE works by harnessing the latent polarisation of p-H₂ in the form of metal bound hydride ligands, and their hyperpolarisation is transferred into the magnetically active nuclei of a weakly bound substrate (14-16) via the small J-couplings that connect them, as quantified by Tessari. (17) Ligand exchange then enables the build-up of a pool of hyperpolarised substrate molecules in solution as detailed in Scheme 1. (18) Remarkably, ¹H polarisations of 50% have been achieved by this route, with ¹⁵N values of 20% seen. In-high-field radio frequency (rf.) transfer has achieved this effect (19) and superior sequences have been developed (20) we employ spontaneous low-field transfer here to create our hyperpolarised agents in a few seconds. Furthermore, as originally predicted, (14) very recent studies have established that SABRE can be used to produce hyperpolarised singlets (22) with magnetic-state-lifetimes that allow signals to be detected 15 minutes after their formation. Hence the SABRE platform reflects a highly desirable route to hyperpolarisation and the extension here to dramatically improve the range of materials it works with is highly desirable. (22-25 & 38)

One of the most effective precatalysts for this process is Ir(COD)(IMes)Cl (1) [where IMes=1,3-bis(2,4,6-trimethylphenyl) imidazole-2-ylidene, COD=cyclooctadiene] and it typically forms [Ir(H)₂(IMes)(substrate)₃]Cl (2) in protic solvents such as methanol⁸, although neutral Ir(H)₂(Cl)(IMes)(substrate)₂ (3) achieves similar results.⁹

Until recently, the polarisable substrate spectrum for p-H₂ based methods comprises unsaturated organic molecules and substrates that temporarily and simultaneously bind with p-H₂ to a transition metal centre.

Lehmkuhl et al “Hyperpolarising Water with Parahydrogen”, ChemPhysChem 10.1002/cphc.201700750³⁹ describes the hyperpolarisation of bulk water with p-H₂ in the presence of an iridium catalyst and histidine.

The initial purpose of the present study was to demonstrate that amines can be successfully hyperpolarised. Hence we report on the use of ammonia (NH₃), benzylamine (BnNH₂), phenethylamine (PEA) and acetamide (AcNH₂). Rapid proton exchange in protic solvents led to the successful hyperpolarisation of a range of ¹H-transfer receptors such as H₂O, butanol and glucose. We have called this second effect RELAY in accordance with its indirect route. This represents an important tool for biochemical analysis²⁹⁻³² but the success in sensitising both the ¹H and ¹³C NMR profiles of these oxygenates marks a watershed in hyperpolarisation.

### SUMMARY OF THE INVENTION

Thus, according to a first aspect of the invention there is provided a method for the preparation of a hyperpolarised target molecule, wherein said molecule comprises at least one —OH, —NH or —SH moiety, via proton exchange from a polarisable molecule, said method comprising the steps of:

(i) preparing a fluid containing a transfer catalyst; parahydrogen; and a polarisable molecule containing at least one exchangeable proton, such as, —OH, —NH or —SH;

(ii) applying a magnetic field or radio frequency excitation such that hyperpolarisation is transferred from parahydrogen to the polarisable molecule when bound to the magnetisation transfer catalyst;

(iii) separately or simultaneously introducing a target molecule, wherein said target molecule contains at least one —OH, —NH or —SH exchangeable proton, enabling hyperpolarisation transfer via proton exchange with the polarisable molecule.

When the target molecule comprises a non-hydrogenatable hydrocarbon moiety, such as, CH₃CH₂COOH, NH₂CH₂Ph, and the like, hyperpolarisation of the target molecule will occur by the proton exchange RELAY effect.

The target substrate can bind reversibly to the transfer catalyst whilst containing a remote site with exchangeable protons. By way of example only, with a pyridyl amine the NH protons are polarised through the SABRE effect in the first instance. SABRE may be used to polarise a substrate wherein subsequent proton exchange activates the RELAYED hyperpolarisation process. Additionally a remote group might be py-OH or py-P(OH)(Ph), and the like.

When the target molecule comprises a hydrogenatable hydrocarbon moiety, such as, an alkene or alkyne, e.g. —CH₂═CHNH₂, and the like, hyperpolarisation of the target molecule may via a hydrogenative route. Such hydrogenative hyperpolarisation occurs by the hydrogenative addition of para hydrogen with the result that the product contains hyperpolarised protons, e.g. hyperpolarised NH protons.

Thus, hydrogenative PHIP can be utilized to polarise a hydrogenatable substrate, whereby subsequent proton exchange activates the RELAYED hyperpolarisation process.

It will be understood that under hydrogenative hyperpolarisation an unsaturated alcohol or phosphine based carrier such as CH2=CHPMe(OH) might also be employed.

In this aspect of the invention the transfer catalyst, e.g. a metal complex, may add H₂ reversibly. The transfer catalyst may contain a ligand which does not dissociate, whilst containing exchangeable NH or OH protons, examples include PMe₂CH₂CH₂NH₂, PMe₂CH₂CHOHPMe₂ or POH(Ph)₂.

That the initial H₂ addition/elimination step, or the amine carrier loss step, may be achieved by UV irradiation or may be thermal or photochemical in nature.

The hyperpolarisation may be achieved by polarisation transfer after, spin refrigeration, DNP, para-hydrogen induced polarisation (PHIP), SABRE or from a suitable molecule in a singlet state. However, in one particular aspect of the invention the hyperpolarisation is introduced by SABRE and thus, the transfer catalyst is a magnetisation transfer catalyst, especially a SABRE magnetisation transfer catalyst.

There are a number of ways to induce certain nuclei into a hyperpolarised state. The simplest way is to cool the material to very low temperatures in the presence of a magnetic field, which will favour population of the lower energy state in which the spins of the nuclei are aligned with the applied magnetic field. This method is suitable for the production of hyperpolarised monatomic gases such as xenon or helium-3. The polarisation levels of these nuclei have also been increased via the use of laser-based technologies.

In SABRE, a catalyst reversibly binds p-H₂ and the polarisable molecule to transfer dormant spin order from p-H₂ into the substrate via a scalar-coupling framework.

This resulting singlet state of the polarisable molecule will desirably be characterised by a long lifetime in a low magnetic field. Preferably, the resulting singlet state lifetime will be 20 seconds or more, preferably more than 20 seconds or more than 25 seconds or more than 30 seconds. The resulting singlet state lifetime may last one or more minutes.

When a SABRE type process is utilised as the method of hyperpolarisation, a SABRE hyperpolarisation transfer catalyst (e.g. [IrCl(COD)IMes] or a ²H-labelled counterpart or a catalyst may be added to optimise the process in a suitable solvent with the selected singlet state derived agent.

H₂ or parahydrogen (p-H₂) gas may be the selected singlet state derived agent and after being added to the resulting system whilst agitating the system will activate the catalyst through a reaction whose speed may be enhanced by stirring, or shaking. Alternatively, the application of ultrasound may be used as a means of agitation. Hyperpolarisation transfer by replacing the H₂ gas with p-H₂ may be performed to create a hyperpolarised transference complex whilst agitating the system as described herein. The addition of H₂ or parahydrogen (p-H₂) gas to the solvent may take place prior to the solvent system being agitated or may take place concurrent with agitation. Catalyst activation under parahydrogen may take place prior to the final hyperpolarisation transfer step or be part of the hyperpolarisation transfer step.

The catalyst, the hyperpolarisable molecule and the target molecule may each contain appropriate ²H or Cl or O labels to maximise the relaxation times of the nuclear spins that are to be hyperpolarised (e.g. ¹H, ¹³C, ³¹P, ¹⁵N, ²⁹Si or ¹⁹F). The target molecule may contain appropriate ¹³C or ¹⁵N labelling to maximise the proportion of the target molecule that can be created in a hyperpolarised NMR visible form in conjunction with appropriate ²H, O or Cl labelling to extend their magnetic state lifetimes. Furthermore, the hyperpolarisable molecule may contain spin pairs of appropriate ¹H, ¹³C, ³¹P, ¹⁵N, ²⁹Si or ¹⁹F labels to enable the formation of long-lived states (singlet states) between the corresponding spin pairs (e.g. ¹H, ¹³C, ³¹P, ¹⁵N, ²⁹Si or ¹⁹F) within a molecular scaffold that contains appropriate ²H or Cl labelling to extend their lifetime. Long lived states may be created from a variety of spin pairs, including pairs comprising ¹H, ¹³C, ¹⁵N, ³¹P, ²⁹Si and ¹⁹F nuclei. The small molecule transference substrate will generally contain its spin ½ nuclei (e.g. ¹H, ¹³C, ³¹P, ¹⁵N, ²⁹Si or ¹⁹F) at the natural abundance level. In the case where the hyperpolarisable molecule contains pairs, these may be homo-nuclear or hetero-nuclear in nature. Examples, of such pairs include, but shall not be limited to ¹H/¹H, ¹H/¹³C, ¹H/¹⁹F, ¹H/¹⁵N or ¹³C/¹³C or any other combination of spin one half nuclei.

Hyperpolarisation will be transferred from parahydrogen into the polarisable molecule in an optimised magnetic field to create a strongly hyperpolarised response. This may be subsequently converted into a singlet state across the spin-pair if desired. This conversion may occur spontaneously or may be promoted by radio frequency excitation. It will be understood that a mixture of transfer catalysts may be included in the method of the invention.

The magnetic field can be changed to focus or improve the efficiency of hyperpolarisation transfer. The type of magnetic states required in this process may be ultra-low magnetic fields, e.g. <<1 G (<10⁻⁶ T) which can spontaneously hyperpolarise the said singlet state. A change in magnetic field can be used to control which substrates in a mixture gain signal in order to introduce selectivity, while varying the field during transfer step to enhance the signal from all substrates.

Molecules containing at least one —OH, —NH or —SH shall also include, for example, metal hydroxides, amines or thiols. Exemplary hydroxides include ⁶LiOH, Al(OH)₃, and the like. Related molecules or complexes containing —OH, —NH or —SH ligands, shall include molecules/complexes with other metals, including ²⁹Si, ⁷⁷Se, ¹¹³Cd, ¹⁹⁹Hg, ¹¹⁷SN, ¹⁹⁵PT, ²⁰⁷PB, ⁵⁷FE, ⁸⁹Y, ¹⁰⁹AG AND ¹⁸³W.

It will be understood that a polarisable molecule containing at least one —OH may comprise, individually or in combination, an alcohol moiety, such as methanol, ethanol, butanol, glucose, alkaloids, prostaglandins, or their salts e.g. NaOCH₃; NaOH; or a P—OH group, such as PO(OH)₃, or their salts e.g. PO(OH)₂(ONa), such as those P—OH groups found in DNA or adenosine triphosphate; or acid functionalities, such as HCOOH, CH₃COOH, CH₃CH₂COOH, CH₃COCOOH, or their salts e.g. NaOOCCH₃; and the like.

It will also be understood that a polarisable molecule containing at least one —NH may optionally comprise an amine or amide moiety or their congeners. Thus, a polarisable molecule containing at least one —NH may comprise, individually or in combination, a primary, secondary or tertiary amine, such as NH₃, NH₄⁺, NH₂Ph, NH₂CH₂Ph, NH₂CH₂HCH₂CH₂Ph; or an amide, such as NH₂COCH₃ or NH₂CONH₂; and the like.

In one aspect of the invention the polarisable molecule contains at least one —OH moiety as herein defined.

In one aspect of the invention the target molecule contains at least one —OH moiety as herein defined.

In another aspect of the invention the polarisable molecule contains at least one —NH moiety as herein defined.

In another aspect of the invention the target molecule contains at least one —NH moiety as herein defined.

In another aspect of the invention the polarisable molecule contains at least one —SH moiety as herein defined.

In another aspect of the invention the target molecule contains at least one —SH moiety as herein defined.

When either the polarisable molecule or the target molecule comprises a molecule containing at least one —NH moiety, the pKa of the molecule, e.g. the amine or amide, can be varied to control the efficiency of proton exchange and/or hyperpolarisation exchange.

The target molecule containing an exchangeable proton may comprise an —OH moiety, an —NH moiety or an —SH moiety as herein defined. Generally, the target molecule containing an exchangeable proton will be different to the polarisable molecule. It will be understood that a mixture of target molecules may be included in the method of the invention.

The target molecule may contain at least one —OH, which may comprise, individually or in combination, an alcohol moiety, such as methanol, ethanol, butanol, glucose, alkaloids, prostaglandins, or their salts e.g. NaOCH₃; NaOH; or a P—OH group, such as PO(OH)₃, or their salts e.g. PO(OH)₂(ONa), such as those P—OH groups found in DNA or adenosine triphosphate; or acid functionalities, such as HCOOH, CH₃COOH, CH₃CH₂COOH, CH₃COCOOH, or their salts e.g. NaOOCCH₃; and the like.

Illustrative examples of target molecules which may be hyperpolarised via this route include, but shall not be limited to:
(i) R—OH or RO⁻ (wherein there is a suitable counter ion such as, but not limited to, Na⁺ or K⁺); wherein R represents alkylC₁₋₂₀, aryl, sugars, glycerol, vinyls, diols, cholesterol, choline, and the like;
(ii) R′COOH or R′COO⁻ (wherein there is a suitable counter ion such as, but not limited to, Na⁺ or K⁺); R′ represents H, alkyl_{C1-20}, aryl, vinyls, or any combination thereof, exemplified by acetic acid, acetate, pyruvate, pyruvic acid, an amino acid, a protein, an enzyme;

(iii) HOP(O)(R)(R′) wherein R and R′, which may be the same or different, each represents H, alkyl_{C1-20}, aryl, etc., such as part of a DNA base pair, strand or RNA or adenosine triphosphate;

(iv) HOBRR′ wherein R and R′, which may be the same or different, each represents H, alkylC₁₋₂₀, aryl, etc., such as part of a borate; and

(v) an inorganic or main group hydroxide such as LiOH, Al(OH)₃ or Ca(OH)₂ and the like or materials taken from the family: ²⁹Si, ⁷⁷Se, ¹¹³Cd, ¹⁹⁹Hg, ¹¹⁷Sn, ¹⁹⁵Pt, ²⁰⁷Pb, ⁵⁷Fe, ⁸⁹Y, ¹⁰⁹Ag or ¹⁸³W.

The target molecule may contain at least one —NH may optionally comprise an amine or amide moiety. Thus, a polarisable molecule containing at least one —NH may comprise, individually or in combination, a primary, secondary or tertiary amine, such as NH₃, NH₄⁺, NH₂Ph, NH₂CH₂Ph, NH₂CH₂HCH₂CH₂Ph; or an amide, such as NH₂COCH₃ or NH₂CONH₂; and the like.

Illustrative examples of target molecules which may be hyperpolarised via this route include, but shall not be limited to:
(i) NR′R″R′″ wherein R′, R″ and R′″, which may be the same or different, each represents H, alkyl_{C1-20}, aryl, base pair, etc. and combined in structures like glutamine, glutamate and GABA;
(ii) NR′R″COR′″ wherein R′, R″ and R′″, which may be the same or different, each represents R′, R″ or R′″═H, CH₃, alkyl, aryl, vinyl, or any combination exemplified by acetamide, urea, glutamine, glutamate, and the like;
(iii) carbamates and carbazides;
(iv) platinum derived cancer drugs, which include, but shall not be limited to cisplatin, carboplatin, nedaplatin, oxaliplatin, triplatin, satraplatin; and the like; and
(v) cancer drugs containing an acetamide group, which include, but shall not be limited to, taxanes such as paclitaxel, docetaxel, cabazitaxel; and the like.

Hyperpolarisation of cancer drugs which have an exchangeable proton, such as cisplatin, carboplatin, paclitaxel and docetaxel may be advantageous in determining impurities in the drugs.

The target molecule may contain at least one —SH may optionally comprise a thiol or a thioamide moiety.

Illustrative examples of target molecules which may be hyperpolarised via this route include, but shall not be limited to:
(i) HSR wherein R represents H, alkyl_{C1-20}, aryl, vinyls, or any combination thereof; and
(ii) thioamides, thioacids, thioureas and xanthates.

A magnetisation transfer catalyst provided with at least one suitable ligation site enabling it to interact with one or more small molecule transference substrates.

The hyperpolarisation transfer catalyst will usually comprise a transition metal complex, for example comprising a metal atom selected from, but not limited to, Ru, Rh, Ir, W, Pd and Pt. In a particular aspect of the present invention a hyperpolarisation transfer catalyst may comprise an iridium based catalysts.

Preferred (SABRE) hyperpolarisation transfer catalysts are thus described in our co-pending application No. PCT/GB2009/002860. Such catalysts include, for example, [IrCl(COD)(IMes)] and analogues thereof, (in which COD is cycloocta-1,5-diene). Alternatively, the (SABRE) hyperpolarisation transfer catalyst may comprise a ²H-labelled counterpart of [IrCl(COD)(IMes)] or a catalyst optimised to work in the non-aqueous phase with the selected substrate.

Generally, an iridium magnetisation transfer catalyst will include iridium with at least one N-heterocyclic carbene (NHC) ligand.

Examples of such N-heterocyclic carbenes include, but shall not be limited to:

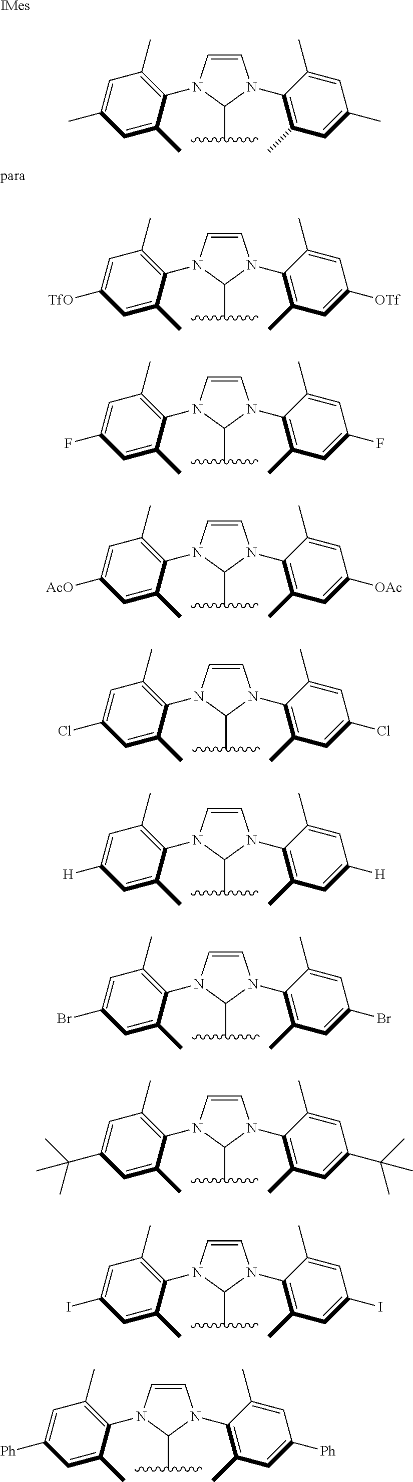

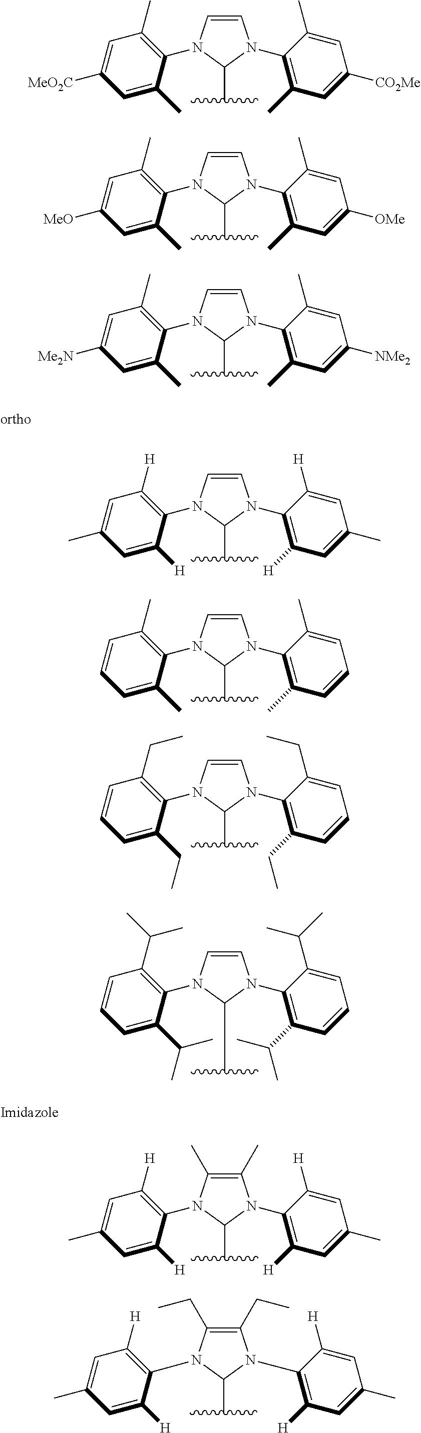

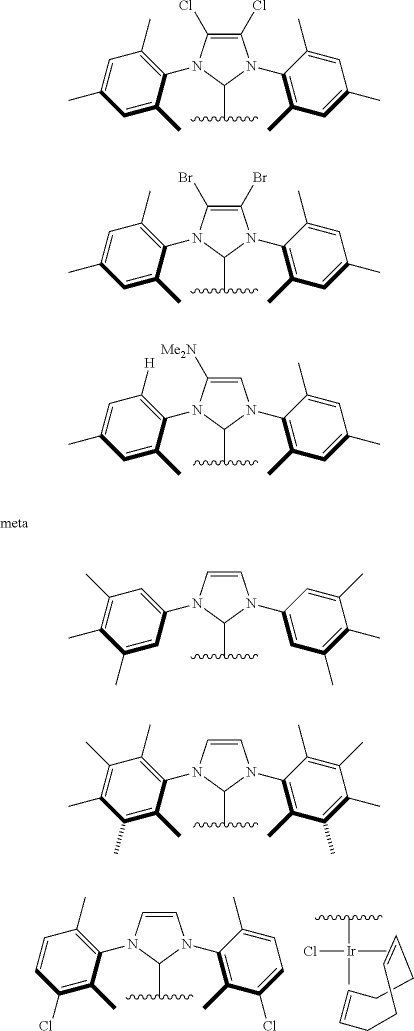

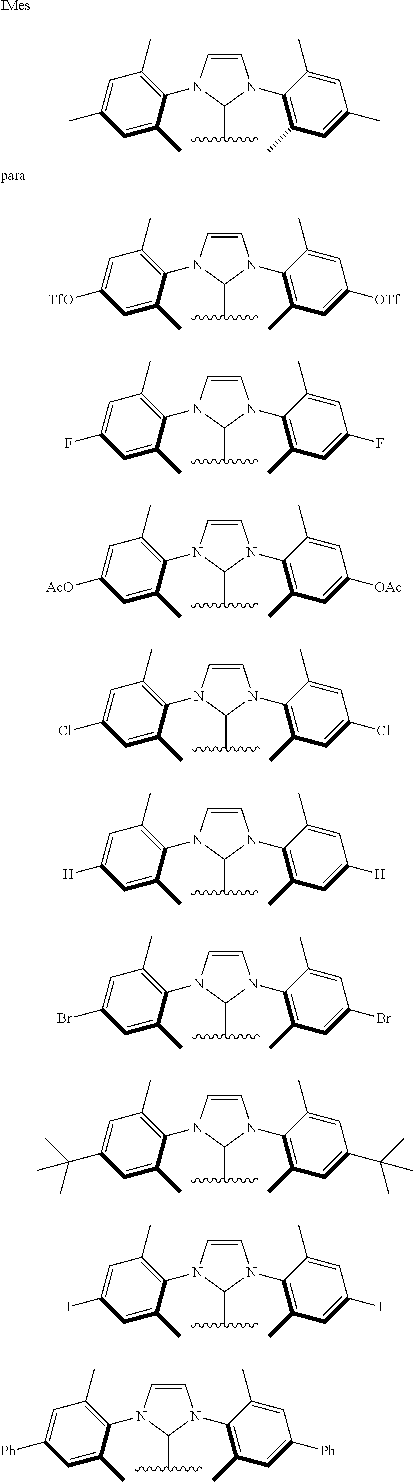

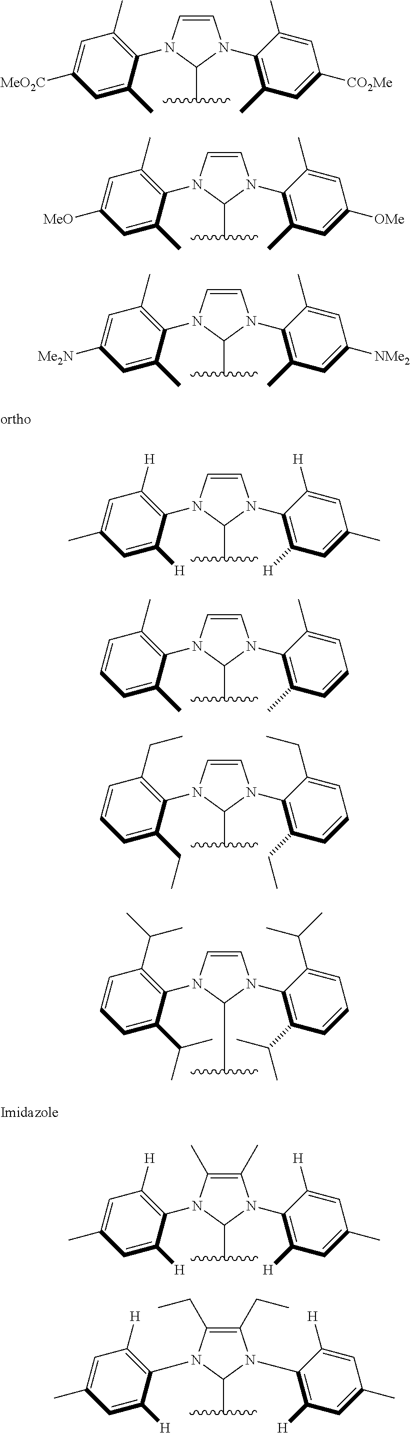

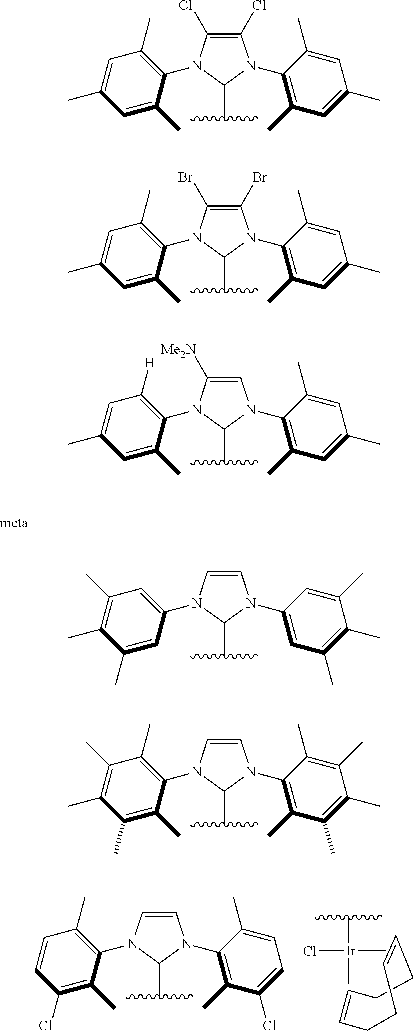

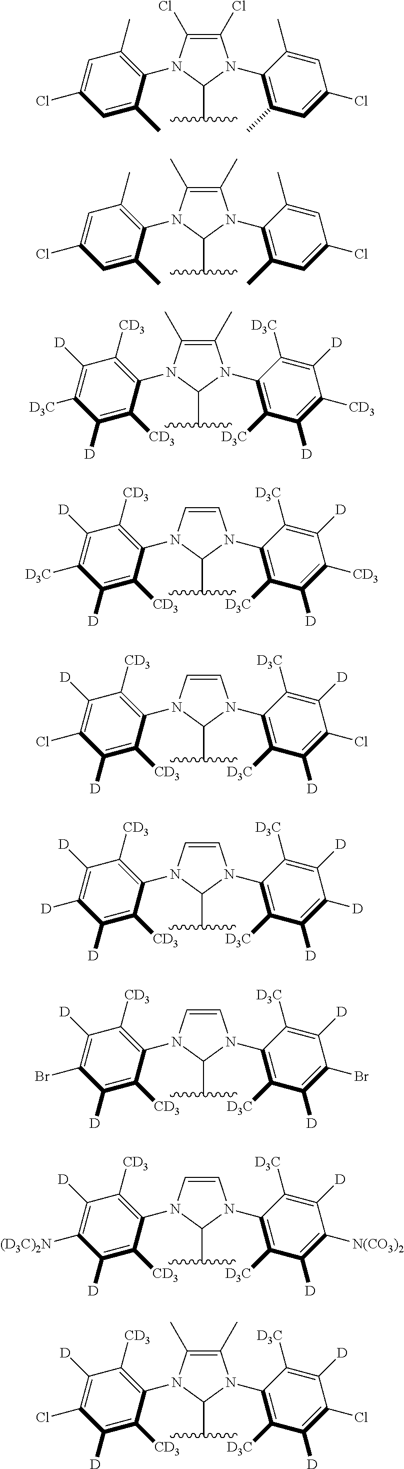

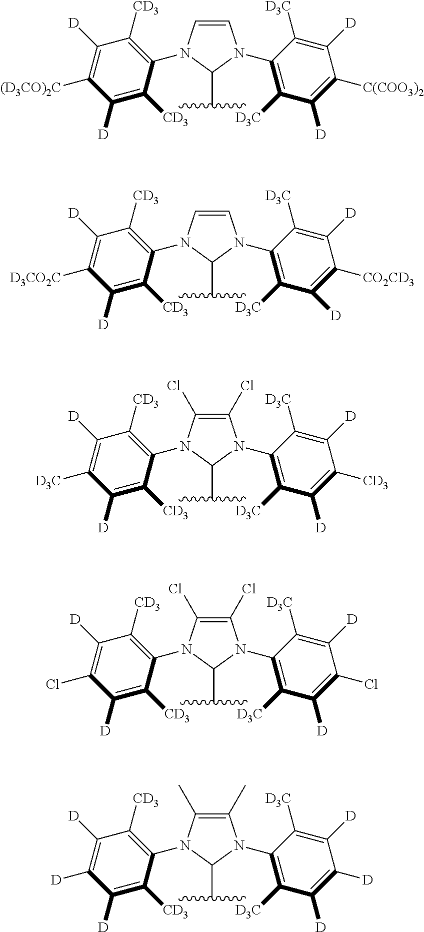

The use of variants of the catalyst [(Ir(H)₂(IMes)(amine)₃]Cl or [(Ir(H)₂(IMes)(amide)₃]Cl where in the ligands are varied can be used to control the efficiency of hyperpolarisation transfer in the first step.

The transfer catalyst may be designed to produce an optimal lifetime and coupling framework for hyperpolarisation transfer under these conditions. It will be understood that a mixture of transfer catalysts may be included in the method of the invention.

These species are often referred to as precatalysts because they are stable and become active during the catalytic process, in this case through their reaction with the small molecule transference substrate and H₂.

A variety of solvents may be used in preparing the fluid required for the method of the present invention. Such solvents will generally be organic solvents and may comprise polar or non-polar solvents. Non-protic solvents are preferred. Such solvents include, but shall not be limited to CH₃OH, CH₃CH₂OH, CH₂OH, CH₂Cl₂, CHCl₃, THF, DMF, nitromethane, alkanes and aromatic hydrocarbons, such as benzene or toluene; the deuterated counterparts of any of the aforementioned solvents. Selection of an appropriate solvent may be used to control one or more of the steps herein defined in the method of the invention.

According to a further aspect of the invention a biphasic element may be introduced into the solvent in order to separate the hyperpolarised target molecule from the transfer catalyst.

The introduction of a biphasic element may comprise preparing a fluid containing two separate components, for example, wherein a first solvent is a polar solvent, e.g. DMSO and a second solvent is an immiscible co-solvent e.g. a non-polar solvent, such as, toluene, chloroform or dichloromethane. The ratio of solvent phases can be selected to:
(i) maximise the degree of target hyperpolarisation; and/or
(ii) maximise the speed of phase separation.

When required an aqueous solvent mixture combination may be used to maximise the relaxation time of the hyperpolarised target molecule in the solution by:
(i) employing D₂O;
(ii) employing a D₂O/H₂O mixture of suitable proportion e.g. 1:1; and/or
(iii) adding a further co-solvent to an appropriate aqueous phase such as ethanol or d₆-ethanol.

When SABRE hyperpolarisation is used, a SABRE hyperpolarisation transfer catalyst (e.g. [Ir(Cl(COD)(IMes)] or a ²H-labelled counterpart or a catalyst optimised to work in the polar phase with the selected singlet state derived substrate). An —NH moiety, e.g. an amine or amide, can be selected to remain in the polar phase of the mixed solvent system.

When a mixed solvent system is used a solvent phase-separation promoter e.g. NaCl or NaO₂CCH₃ or NaOH or NaHCO₃ or Na₂CO₃ or ethanol, at a suitable concentration may be added to the system.

The concentration of the phase-separation promoter may be an amount suitable to:
(i) achieve physiological conditions;
(ii) vary the solutions pH to achieve optimal SABRE;
(iii) optimise organic phase extraction; and/or
(iv) optimise the speed of phase-separation.

Any known phase-separation promoter may be used. Desirably such a phase-separation promoter will be suitable for in vivo use and therefore should be suitable to achieve physiological conditions. In addition, the phase-separation promoter should be suitable to withstand variations in pH which may be desirable to achieve optimal SABRE. Selection of the phase-separation promoter may also be desirable to optimise organic phase extraction; and/or to optimise the speed of phase-separation.

Examples of phase-separation promoters include alkali metal salts, such as sodium or potassium salts; or alkaline earth metal salts, such as calcium. Alkali metal salts are preferred, such as NaCl, or NaO₂CCH₃, NaOH, NaHCO₃ or Na₂(CO₃). A further phase-separation promoters may comprise an alcohol such as ethanol.

The amount of phase-separation promoters may vary depending, inter alia, upon the nature of the phase-separation promoters, the nature of hyperpolarisation target, etc. When the aim is to create a biocompatible system, NaCl or KCl may be used as a phase-separation promoter to produce a saline or saline-like solution. Therefore, the amount of the phase-separation promoter may vary depending upon, inter alia, the nature of the phase-separation promoter. Generally, the phase-separation promoter may be from about 0.33% w/v to about 9% w/v. However, it will be understood by the person skilled in the art that more or less of the phase-separation promoter may be included, as required.

The hyperpolarisation transfer may be performed with p-H₂ to create a hyperpolarised target molecule whilst agitating the biphasic solvent as herein described.

An appropriate amount of time may be allowed to enable the two solution phases to separate.

The result of the hyperpolarisation process described herein is that the magnetic resonance signature of the —OH, —NH or —SH bearing moiety contains a hyperpolarised response in its ¹H, ¹⁹F, ¹³C, ³¹F and ¹⁵N nuclei. This is achieved through transfer of hyperpolarisation when the polarisable molecule is bound to the transfer catalyst, which locates a lone pair of electrons of the —OH, —NH or —SH moiety within the bonding framework of this complex. This process reduces the rate of any hydrogen exchange processes it is involved in thereby improving hyperpolarisation transfer efficiency which can be further optimised by catalyst or solvent variation.

Furthermore, the use of ²H or ¹⁵N labelling in the polarisable molecule may be used to improve their relaxation times and increase the levels of detectable hyperpolarisation in them and the target molecule(s).

The polarisable molecule is then released from the metal in a hyperpolarised form and its hyperpolarised ¹H, ¹³C, ³¹P, ¹⁹F and ¹⁵N response can be detected.

In the presence of a target molecule as herein defined, e.g. that contains an exchangeable proton, hyperpolarisation can be transferred into the ¹H, ¹⁹F, ¹³C, ³¹F and/or ¹⁵N nuclei of the target molecule.

The route to hyperpolarisation transfer from the polarisable molecule to the target molecule can be via proton exchange. By way of example only, if the polarisable molecule is ethanol, CH₃CH₂OH, such that the OH of the alcohol is hyperpolarised; and the target molecule is ammonia, NH₃; the mechanism of proton transfer may involve the formation of CH₃CH₂O⁻ and NH₄⁺ species. Subsequent reformation of CH₃CH₂OH and NH₃ will result in the creation of hyperpolarised CH₃CH₂OH and NH₃. Hyperpolarisation transfer from the proton then hyperpolarises the ¹³C and/or ¹H response of the CH₃CH₂OH. An analogous mechanism will operate for hydroxy acids.

It will be understood by the person skilled in the art that the term “proton exchange” may include establishment of a hydrogen bonding interaction between the polarisable molecule and the target molecule during the hyperpolarisation transfer step.

In the example given, a further route to hyperpolarisation may take place within the charge separated oxy anion, CH₃CH₂O⁻ and the ammonium, NH₄⁺, forms of the polarisable and target molecules, if they are solvated within a solvent cage.

A further form of this process may involve the utilization of parahydrogen enhanced hydride ligands of the transfer catalyst/complex, such as [(Ir(H)₂(IMes)(NH₃)₃]Cl being deprotonated by NH₃ to form NH₄⁺, this again will place a hyperpolarised proton into NH₃. Subsequent exchange with a target molecule then allows transfer according to the pathways herein described.

In a further variant of the catalyst, reversible H₂ addition leads to a hyperpolarised metal complex which contains a pendant group with exchangeable protons such as MCH₂NH₂, MCH₂OH which once polarised undergoes RELAY without formal loss of the whole ligand.

In a further variant of the catalyst reversible H₂ addition leads to a metal complex with an acidic proton that can either form a hydrogen bond for RELAY without formal loss or be removed via deprotonation through the interaction with a suitable base such as NH₃, NaOH or Cs₂CO₃.

Through the process described herein the NMR or MR response of the target molecule can be increased so that it is readily detectable in a high resolution or imaging experiment.

Furthermore, the use of ²H or ¹⁵N labelling in the target molecule may be used to improve their relaxation times and increase the levels of detectable hyperpolarisation.

The target molecule will generally:
(i) contain spin ½ nuclei (e.g. ¹H, ¹³C, ³¹P, ¹⁵N or ¹⁹F) at the natural abundance level;
(ii) contain appropriate ²H or Cl labels to maximise the relaxation times of the nuclei spins that are to be hyperpolarised (e.g. ¹H, ¹³C, ³¹P, ¹⁵N or ¹⁹F);
(iii) contain appropriate ¹³C or ¹⁵N labelling to maximise the proportion of the target that can be created in a hyperpolarised NMR visible form in conjunction with appropriate ²H or Cl labelling to extend their magnetic state lifetimes; and
(iv) contain pairs of appropriate ¹H, ¹³C, ³¹P, ¹⁵N or ¹⁹F labels to enable the formation of long-lived states (singlet states) between the corresponding spin pairs (e.g. ¹H, ¹³C, ³¹P, ¹⁵N or ¹⁹F) within a molecular scaffold that contains appropriate ²H or Cl labelling to extent their lifetime.

The hyperpolarisation target molecule may reflect a complex biomolecule containing exchangeable protons such as an enzyme, a protein, an alkaloid, an oligosaccharide or strand of DNA, RNA or adenosine triphosphate. After proton exchange the target biomolecule will become sensitised to NMR or MRI detection. This approach is therefore suited to the characterisation of large molecules and the probing of drug binding/active site conformations, dynamics and folding.

The use of NH₃, NH₂Ph, NH₂CH₂CH₂Ph (etc.) and related amines or amides (e.g. CH₃CONH₂) and their ²H or ¹⁵N labelled counterparts can be used to control the efficiency of hyperpolarisation transfer in the first step. This is a result of the metal complexes reactivity which can be optimised for specific solvent, cost, pressure of p-H₂ and time of activation.

The temperature can be changed to focus or improve the efficiency of hyperpolarisation transfer.

In one aspect of the present invention the polarisable molecule and the target molecule may be present at point (i), i.e. when the polarisable molecule is hyperpolarised. We also note, more than one target may be present.

The transfer catalyst will usually comprise a transition metal complex, for example comprising a metal atom selected from, but not limited to, Ru, Rh, Ir, W, Pd and Pt. The transfer catalyst will usually comprise one or more ligands in addition to the ligand comprising the hyperpolarisable nuclei. These one or more other ligands may comprise organic or inorganic ligands and may be mono-, bi- or multidentate in nature. These one or more ligands may play a role in controlling the activity and stability of the metal centre. For example, the one or more ligands may comprise NHC ligands as herein described.

In one embodiment, the transfer catalyst comprises one or more phosphine ligands in addition to the ligand to be hyperpolarised. The transfer catalyst may be attached to a solid support, for example a polymer support. Attachment will usually be made through a ligand which links the metal centre to the support. Suitable linkers are known in the art. For example, the linker may comprise one or more in-chain atoms selected from C, O, N, S, P and Si. The linker may comprise a siloxane moiety for attachment to the support and/or a phosphine moiety for attachment to the metal of the complex. In embodiments, the linker is a group of the following formula: —O—Si(OMe)₂—(CH₂)ₙ—P(Cy)₂—, wherein n is 0 upwards (e.g. 0, 1, 2, 3, 4, 5 or 6) and Cy is cyclohexyl.

For in vivo use an in-line UV probe may be used, if desired, to establish that the concentration of the polarisable molecule is sufficiently low for in vivo injection. This makes full use of the fact that the catalyst is no longer present and therefore unable to promote the relaxation of the agent, thereby maximising longevity of the resulting hyperpolarised signal.

For systems where the catalyst concentration remains too high, a catalyst deactivator may be added, to facilitate catalyst transfer. Examples of suitable catalyst deactivators include, but shall not be limited to, bipyridyl, EDTA and dimethylglyoxime.

An appropriate delivery device may be used to procure the hyperpolarised target molecule for detection by NMR or MRI which can facilitate some or all of the following:

Using an appropriate delivery device to procure the hyperpolarised agent for detection by NMR or MRI which will facilitate some (all) of the following:
(i) after an appropriate amount removing a hyperpolarised sample from the aqueous phase;
(ii) using UV monitoring to assess suitability immediately prior to sample removal or after sample removal;
(iii) using pH monitoring to assess suitability immediately prior to sample removal or after sample removal;
(iv) employing filtration to achieve sterility after sample removal;
(v) injecting or transporting the sample into a target for subsequent detection by NMR or MRI, where the target might be a suitable sample tube, an animal or a human.

According to a further aspect of the invention there is provided a method of producing a hyperpolarised imaging medium, said method comprising the steps of:
(i) preparing a fluid containing a transfer catalyst; parahydrogen; and a polarisable molecule containing at least one —OH or —NH moiety;
(ii) applying a magnetic field or radio frequency excitation such that hyperpolarisation is transferred from parahydrogen to the polarisable molecule when bound to the magnetisation transfer catalyst;
(iii) separately or simultaneously introducing a target molecule(s), wherein said target molecule(s) contains an exchangeable proton, enabling hyperpolarisation transfer via proton exchange;
(iv) optionally separating the hyperpolarised target molecule(s) to provide a hyperpolarised target molecule imaging medium; and
(v) completing NMR or MRI measurements on the system prior to repeating the process for signal averaging.

According to a further aspect of the invention there is provided a pharmaceutically acceptable formulation comprising a solution of a hyperpolarised target molecule, said target molecule containing at least one —OH or —NH moiety, for use as an imaging medium, wherein said hyperpolarised target molecule is prepared by proton exchange from a hyperpolarised molecule containing at least one —OH or —NH moiety, said method comprising the steps of:
(i) preparing a fluid containing a transfer catalyst; parahydrogen; and a polarisable molecule containing at least one —OH or —NH moiety;
(ii) applying a magnetic field or radio frequency excitation such that hyperpolarisation is transferred from parahydrogen to the polarisable molecule when bound to the magnetisation transfer catalyst;
(iii) separately or simultaneously introducing a target molecule, wherein said target molecule contains an exchangeable proton, enabling hyperpolarisation transfer via proton exchange.

In a preferred aspect of the invention the pharmaceutically acceptable formulation comprises a solution of a target molecule in a saline solution of a hyperpolarised target molecule for use as an imaging medium.

According to a yet further aspect of the invention there is provided an imaging medium for in vivo magnetic resonance (MR) detection comprising a hyperpolarised target molecule containing at least one —OH or —NH moiety wherein said hyperpolarised target molecule is prepared by proton exchange from a hyperpolarised molecule containing at least one —OH or —NH moiety, said method comprising the steps of:
(i) preparing a fluid containing a transfer catalyst; parahydrogen; and a polarisable molecule containing at least one —OH or —NH moiety;
(ii) applying a magnetic field or radio frequency excitation such that hyperpolarisation is transferred from parahydrogen to the polarisable molecule when bound to the magnetisation transfer catalyst;
(iii) separately or simultaneously introducing a target molecule, wherein said target molecule contains an exchangeable proton, enabling hyperpolarisation transfer via proton exchange.

We have demonstrated how it is possible to use parahydrogen to sensitise the MR response of a range of molecules that contain the four common and very important functional groups NH₂, OH, NH₂CO, COOH, COO⁻ and P(O)(OH)₃. We achieve this by taking a simple amine such as ammonia and reacting it with [IrCl(COD)(IMes)] (1) [where IMes=1,3-bis(2,4,6-trimethylphenyl) imidazole-2-ylidene, COD=cycloocta-1,5-diene] and p-H₂. For this amine, [Ir(H)₂(IMes)(NH₃)₃]Cl (2-NH₃) rapidly forms in methanol or dichloromethane solution. At 243 K, in methanol-d₄, 2-NH₃ exhibits a hydride resonance at δ-23.2 in a ¹H NMR spectrum that was recorded at 9.4 T, which rapidly separates into several components as H-D exchange proceeds to form an array of NH₃ isotopologues. Notably, a hyperpolarised NMR signal is seen at δ 5.06 for the exchangeable proton of CD₃OH (8-fold gain) alongside a weaker signal at δ 2.19 that is enhanced by just two-fold. However, when the same reaction is monitored in dry dichloromethane-d₂ solution, with a 10-fold excess of NH₃ relative to 1 at 298 K, the hydride signal of 2-NH₃ appears as a simple singlet at δ −23.8, alongside a broad response at δ 0.47 for NH₃ with the equatorial and axial NH₃ ligands of 2-NH₃ yielding signals at δ 2.19 and 2.88 respectively. A series of 2D ¹H-¹⁵N measurements were used located the corresponding ¹⁵N signals for 2-NH₃ at δₐₓᵢₐₗ −47.8 and δ_{equ} −35.5 and confirm these assignments.

Now, when a 2 bar pressure of p-H₂ is employed at 298 K in dichloromethane-d₂, the ¹H NMR signal for free NH₃ shows a ˜10-fold enhancement per proton after hyperpolarisation transfer in a 60 G field according to Scheme 1, with the bound NH₃ ligand signal at δ 2.19 showing a 3-fold enhanced response. These observations confirm that 2-NH₃ undergoes SABRE and is involved in realising the hyperpolarised free amine return. Hydrogen atom exchange then accounts for the observation of the enhanced δ 5.06 signal in methanol, which is actually the exchange averaged response of CD₃OH and NH₃. We therefore added a 5% loading of H₂O to a new dichloromethane solution of 2-NH₃ which contained a 23-fold excess of NH₃. The corresponding H₂O based signal appears at δ 1.88 and when such a sample is under 3 bar of p-H₂, the free NH₃ signal gain under SABRE increases to 40-fold per proton whilst the equatorial ligand signal gain proved to be 85-fold per proton with the H₂O signal gain being 75-fold gain per proton. Exchange spectroscopy measurements confirmed that the free and equatorially bound NH₃ ligand are in chemical exchange, with the observation of further exchange peaks between free NH₃ and H₂O demonstrating the rapid chemical transfer of the exchangeable protons between them. Clearly, when bound, NH₃/H₂O proton exchange is suppressed as the nitrogen lone pair is involved in bonding to the metal and consequently ¹H-SABRE occurs to hyperpolarise the amine. Following its dissociation, chemical exchange then leads to observation of hyperpolarisation in the effective proton response of H₂O or HOCD₃.

We showed that the SABRE hyperpolarisation of NH₃ could be transferred into the ¹H and ¹³C responses of methanol, ethanol, or 1-butanol by doping three solutions with one microliter of each. Amazingly, strong hyperpolarisation was seen in their ¹H (methanol, OH, 630-fold, Me, 505-fold per proton) and ¹³C responses as illustrated in FIG. 20 for 1-butanol. This result illustrates that what we have called a SABRE-RELAY effect enables the easy hyperpolarisation of these alcohol, with hyperpolarised states being created for all of the NMR active nuclei that lie all along their carbon skeletons.

We therefore looked at the effect of doping a series of H₂O/NH₃ containing samples with acetic acid, sodium acetate, ethanoic acid, sodium ethanoate, pyruvic acid or sodium pyruvate. Strong hyperpolarisation was again observed in all cases for salt based solutions for their proton resonances, and when they were ¹³C labelled at the carbonyl position a hyperpolarised ¹³C response was readily visible. The acid samples showed similar effects initially, but as might be expected, their ammonium salts precipitated over time and stopping the hyperpolarisation effect. By controlling the pH hyperpolarised signals for the acids can be viewed. It is advantageous to use pH to control what is enhanced and degree of enhancement.

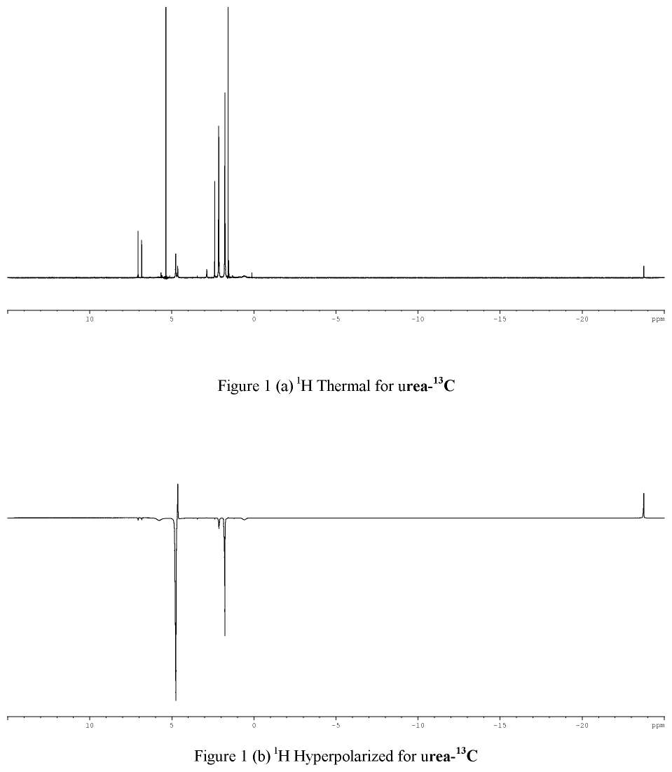

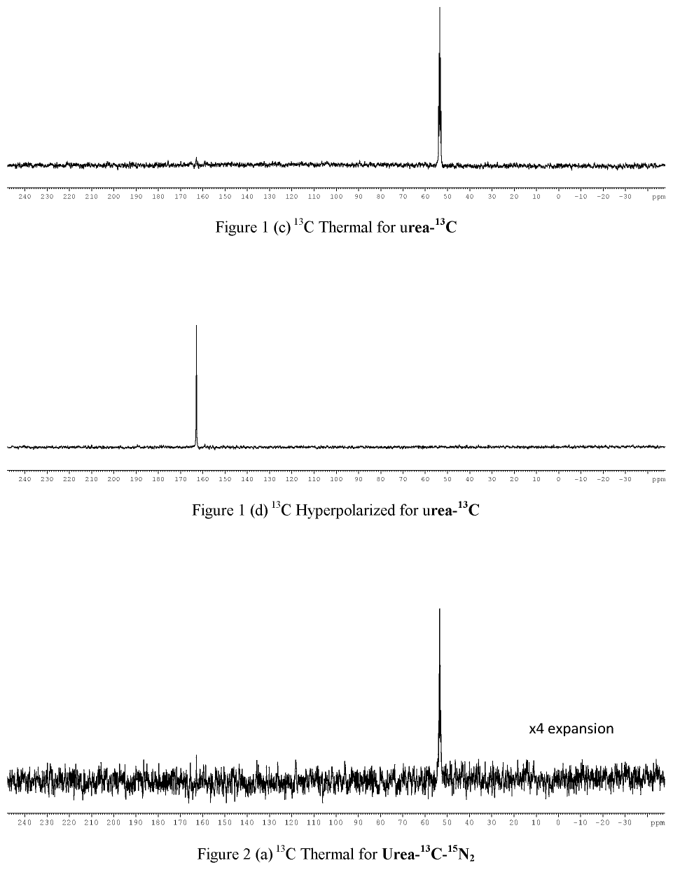

Next we examined the amines benzylamine and phenylethylamine and the amides acetamide, urea and methacrylamide. We found that both benzylamine and phenylethylamine were hyperpolarised readily without the need for added ammonia, and ultimately demonstrated that they could be used as hyperpolarisation carriers in the same way as demonstrated for ammonia. In contrast, the NH₃/H₂O carrier enabled acetamide, urea and methacrylamide to show substantial signal gains in their ¹H, ¹³C and ¹⁵N responses when tested with p-H₂ as detailed in FIG. 1 (and the supporting information). This time, the NH₂ group of acetamide showed a 170-fold gain per proton while the CH₃ group gain was 80-fold per proton and the added water returned a 130-fold gain per proton. We note that in these measurements, 2-NH₃ remained visible as the sole iridium based product and therefore plays a common role in SABRE-RELAY in all cases here. We have therefore illustrated a general route wherein optimisation of the process can be achieved for a given target(s) by varying the amine, H₂O or HOR loading, the solvent and the catalyst.

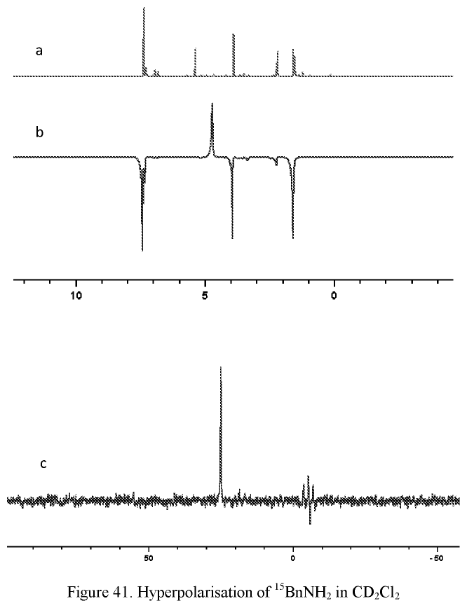

It is possible to track these changes when benzylamine (BnNH₂) or phenethylamine (PEA) are used alone, [Ir(H)₂(IMes)(NH₂Bn)₃]Cl (2-BnNH₂) and [Ir(H)₂(IMes)(NH₂CH₂CH₂Ph)₃]Cl (2-PEA) formed readily in CD₃OD and dichloromethane solution. In methanol-d₄, the free NH₂ signal of 2-BnNH₂ shows just a two-fold signal enhancement whilst the CH₂ group records a 51-fold gain and the 5 aromatic protons a 20-fold gain (per proton) for a sample with a 10-fold excess of BnNH₂ relative to 1. The δ 5.06 signal showing an improved signal gain of 50-fold per proton initially. However, over time, the H/D exchange with the solvent now results in the formation of BnND₂ and PEA-ND₂ which acts to suppress SABRE. In contrast, when dichloromethane-d₂ is used, the signal gains for free BnNH₂ increase to 72, 53 and 170-fold (aromatic) respectively per proton and remain visible for days if the solution is reactivated with p-H₂. 2-BnNH₂ was fully characterized in CD₂Cl₂ solution (see supporting information) and returns a diagnostic hydride signal at δ −23.95. Furthermore, SABRE transfer to the bound ¹H, ¹³C and ¹⁵N responses BnNH₂ of occurs as revealed in FIG. 41. Interestingly, the NH₂Bn ligand which lies trans to hydride actually yields inequivalent responses for its NH₂ protons at δ 4.92 and 2.30, and its CH₂ protons at δ 3.60 and 3.18. In contrast, the corresponding axial ligand yields single responses for these signals at δ 4.24 (NH₂) and δ 3.83 (CH₂) respectively which are not hyperpolarised. This is due to the fact that rotation about these Ir—N bonds results in hydride and axial ligand proton equivalence, whilst such a process acts to maintain an up/down distinction for those of the equatorial ligand.

When the aliphatic chain of BnNH₂ is extended from methylene to ethylene, the new substrate phenethylamine (PEA) is again observed to perform well in dichloromethane-d₂, with the corresponding NH₂ signal gain being 108-fold per proton for a 10-fold loading at 298 K. Polarisation transfer across the ethylene bridge into the phenyl group is again observed such that signal gains of 50-(NCH₂), 45-(CH₂), 92 (ortho), 50 (meta) and 20-(para) fold result. We note that 2-PEA also forms cleanly, yielding a hydride signal at δ −23.78.

As identified earlier, upon replacing the SABRE-RELAY promoter NH₃ by BnNH₂ or PEA, hyperpolarisation transfer into these agents is observed. In the case of PEA, the efficiency of urea hyperpolarisation is improved whilst that of ¹³C-labelled glucose is reduced. Hence we conclude that amine variation will enable the further optimisation of this approach whilst also allowing the introduction of selectivity into the hyperpolarisation receptor. We have also tested the effect of replacing the imidazole CHCH group with CMeCMe and found that the hyperpolarisation levels double. We have also experimented with a number of different catalysts and have seen further improvements over the enhancements seen with IMes. As a consequence while we have shown here how SABRE-RELAY can be used to hyperpolarise a wide range of materials by harnessing the reversible chemical exchange of hyperpolarised NH protons with those of a partner in a range of amines, amides, alcohols, carboxylic acids, oxalic acids, carbonic acid (including salts of such acids, e.g. salts of oxalic acid, such as Na₂C₂O₄, and salts of carbonic acid, such as NaHCO₃) and phosphates we expect significant improvements to be possible for these already impressive results by catalyst variation.

This approach has the potential to revolutionise NMR as the microliter doped butanol sample was just 9×10⁻⁶ molar and a ¹³C spectrum was successfully recorded in just one observation. Additionally, we know that low field detection of such signals is also possible when such signal gains are harnessed, and hence the potential for employing metabolic phenotyping by NMR in less-advanced areas of the world such as those pioneered by Nicholson may at last be realised. Given the fact that exchangeable protons play a growing role in biochemical NMR analysis we expect this relayed hyperpolarisation transfer result to be of significant interest to biochemists, especially if augmented with a LIGHT-SABRE (20) type approach to enable in-high-field hyperpolarisation transfer. Given that hyperpolarised urea, glucose and pyruvate have proven suitable as diagnostic probes for disease in MRI we expect this low cost route to their hyperpolarisation to be especially important, especially if one considers we have already shown the SABRE approach delivers a continuously hyperpolarised bolus. Furthermore, studies of catalysis using PHIP have made significant contributions to our understanding of an array of chemical transformations. (27-33) We therefore expect these results to unlock similar high sensitivity studies on transfer hydrogenation, (34, 35) hydroamination, (36) and even vitally important N₂ fixation to name but a few important examples. (37)

The invention will now be illustrated by way of example only and with reference to the accompanying drawings, in which:

FIGS. 1 (a)-(d) are the spectra for (a) ¹H thermal, (b) ¹H hyperpolarised, (c) thermal and (d) ¹³C hyperpolarised for urea-¹³C using conditions: [IrCl(COD)(IMes)] (5 mM), Urea-1-¹³C (10 eq.), CD₂Cl₂ (0.6 mL), NH₃ and H₂O (5 μL);

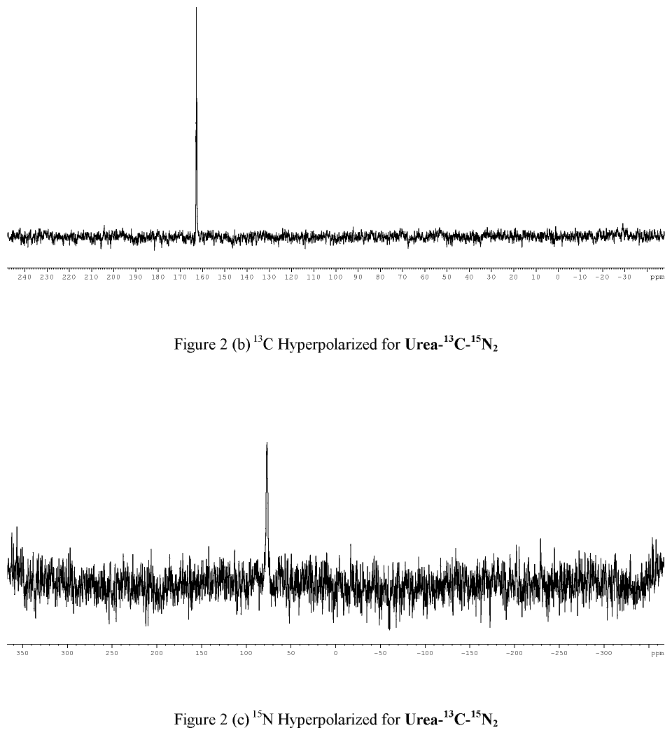

FIGS. 2 (a)-(d) are the spectra for (a) 13C thermal, (b) 13C hyperpolarised, (c) ¹⁵N hyperpolarised for urea-¹³C-¹⁵N₂ using conditions: [IrCl(COD)(IMes)] (5 mM), Urea-¹³C-¹⁵N₂ (10 eq.), CD₂Cl₂ (0.6 mL), NH₃, H₂O (5 μL);

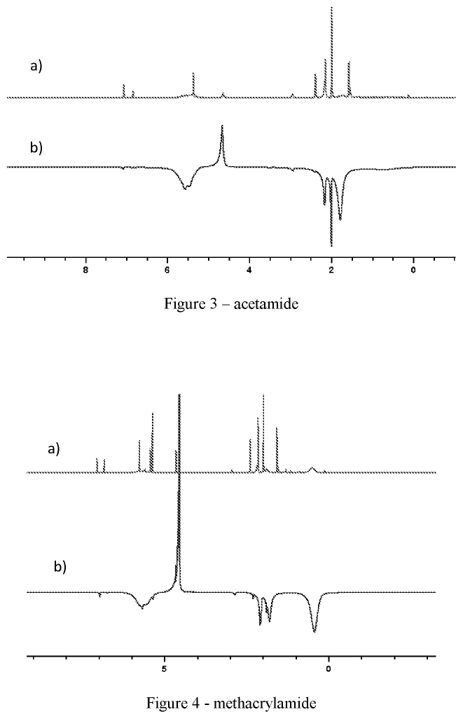

FIG. 3 is the spectra for acetamide using conditions: [IrCl(COD)(IMes)] (5 mM), acetamide (10 eq.), CD₂Cl₂ (0.6 mL), NH₃;

FIG. 4 is the spectra for methacrylamide using conditions: [IrCl(COD)(IMes)] (5 mM), methacrylamide (10 eq.), CD₂Cl₂ (0.6 mL), NH₃;

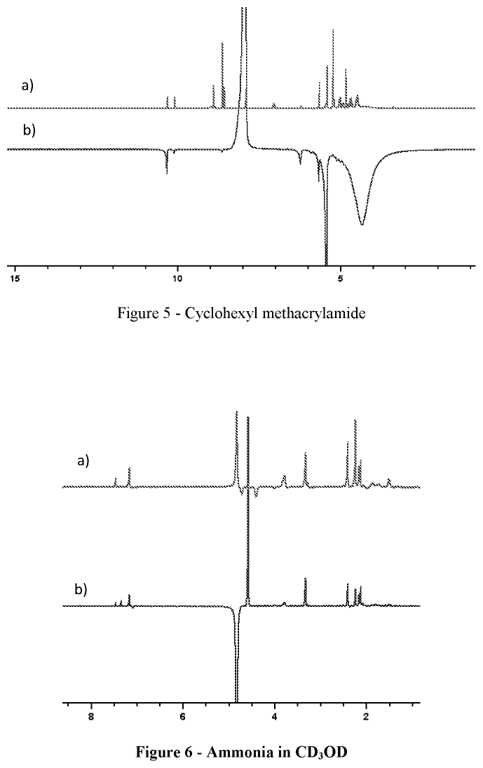

FIG. 5 is the spectra for cyclohexyl methacrylamide using conditions: [IrCl(COD)(IMes)] (5 mM), Cyclohexyl methacrylamide (10 eq.), CD₂Cl₂ (0.6 mL), NH₃;

FIG. 6 is the spectra for Ammonia in CD₃OD: a) ¹H spectrum after first shake under p-H₂ (start of the activation process), b) after 1 min of the activation, the methanol does hyperpolarisation after H/D exchange; using conditions: [IrCl(COD)(IMes)] (5 mM), CD₃OD (0.6 mL), NH₃;

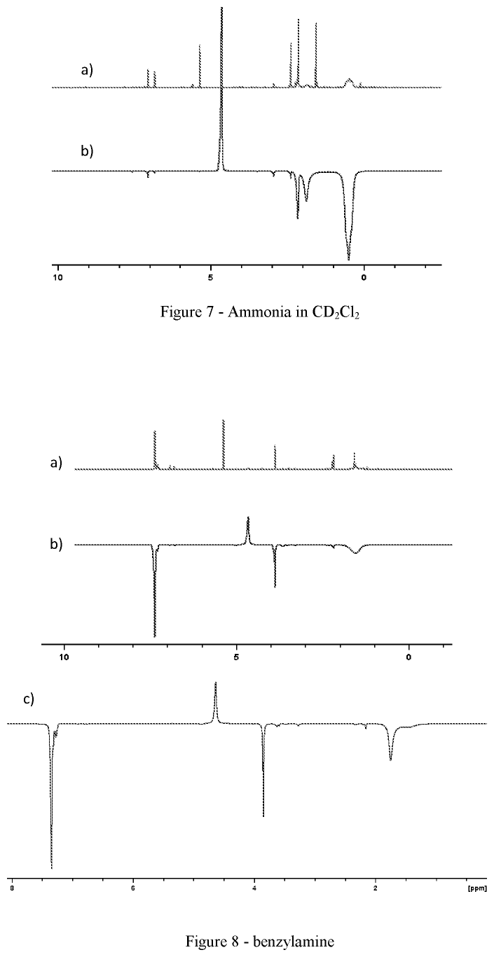

FIG. 7 is the spectra for ammonia in CD₂Cl₂: a) ¹H thermal ×16, b) ¹H SABRE hyperpolarisation (max enhancement 94 fold); using conditions: [IrCl(COD)(IMes)] (5 mM), CD₂Cl₂ (0.6 mL), NH₃;

FIG. 8 is the spectra for benzylamine a) ¹H thermal ×8, b) ¹H SABRE hyperpolarisation (max enhancement 160 fold) and c) after adding 1 μl of D₂O (max enhancement of H₂O 215 fold); using conditions: [IrCl(COD)(IMes)] (5 mM), benzylamine (10 eq.), CD₂Cl₂ (0.6 mL);

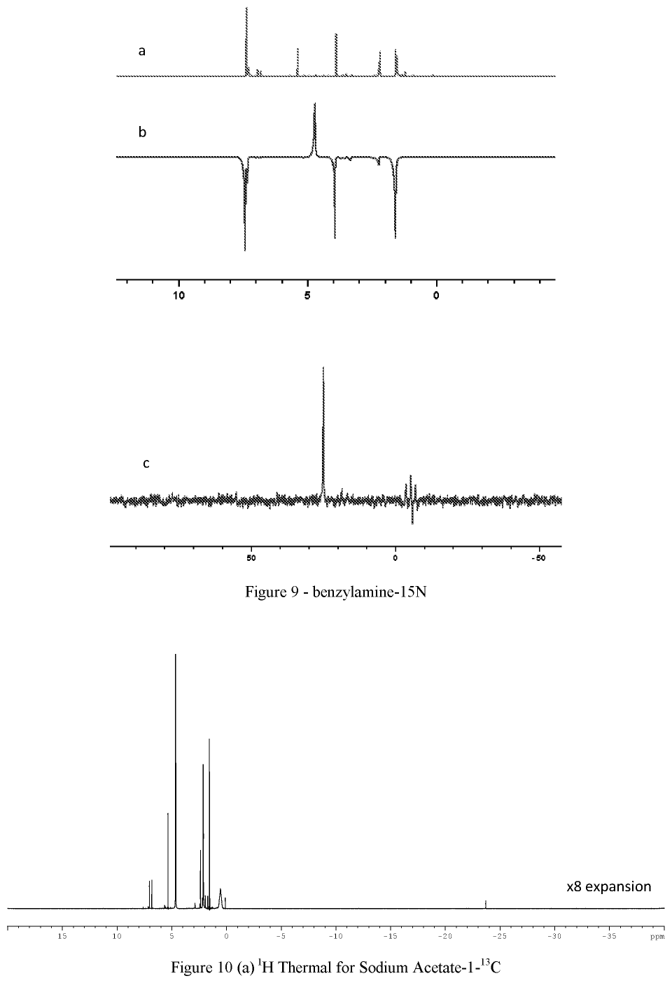

FIG. 9 is the spectra for benzylamine-15N a) ¹H thermal ×4, b) ¹H SABRE hyperpolarisation (max enhancement 60 fold) and c) ¹⁵N hyperpolarisation; using conditions: [IrCl(COD)(IMes)] (5 mM), benzylamine-¹⁵N (10 eq.), CD₂Cl₂ (0.6 mL);

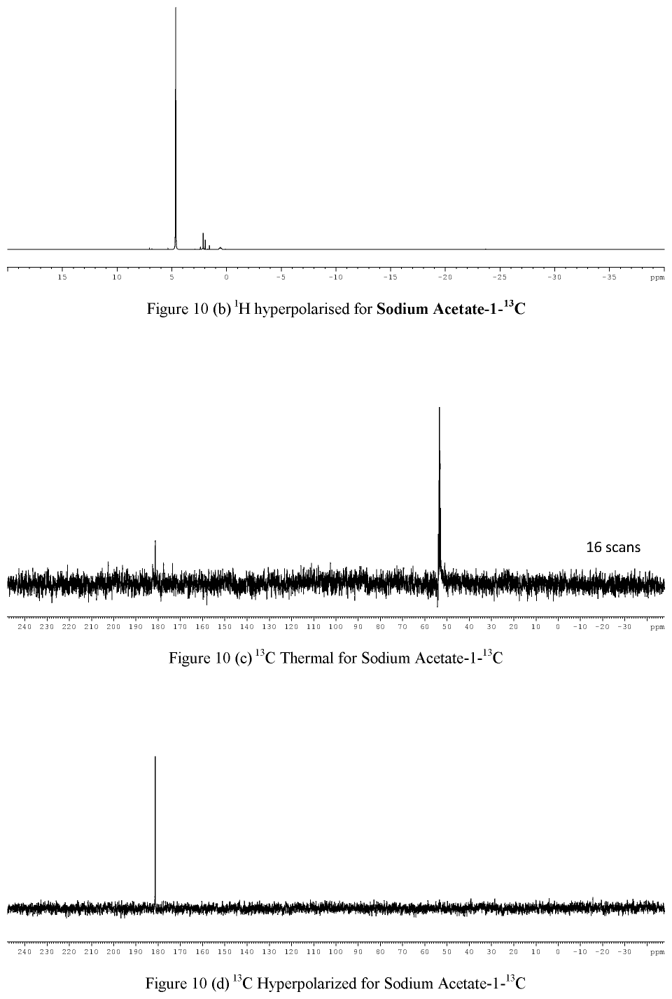

FIGS. 10 (a)-(d) are the spectra for (a) ¹H thermal, (b) ¹H hyperpolarised, (c) ¹³C thermal and (d) ¹³C hyperpolarised for sodium acetate-1-¹³C using conditions: [IrCl(COD)(IMes)] (5 mM), sodium acetate-1-¹³C (10 eq.), CD₂Cl₂ (0.6 mL), NH₃, H₂O (5 μL);

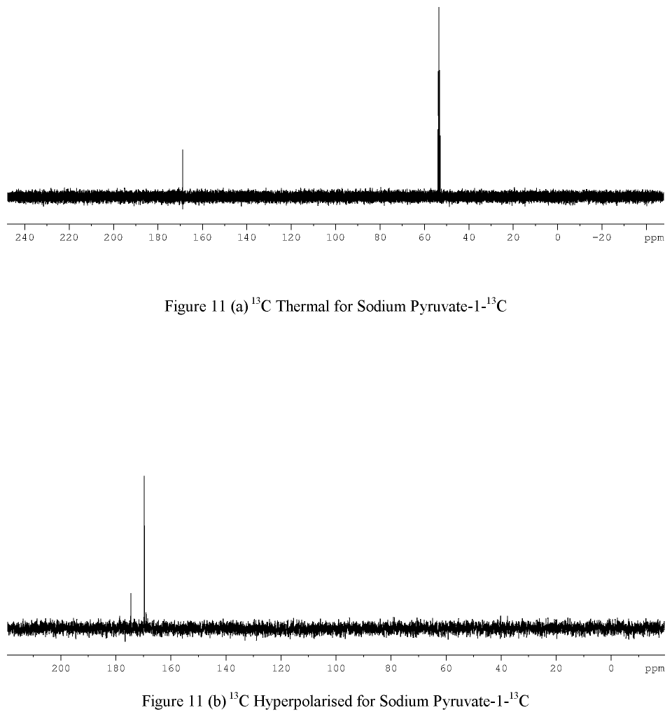

FIGS. 11 (a)-(b) are the spectra for (a) ¹³C thermal and (b) ¹³C hyperpolarised for Sodium Pyruvate-1-¹³C using conditions: [IrCl(COD)(1,3-bis(2,4,6-trimethylphenyl)-4,5-dimethylimidazole)] (5 mM), sodium pyruvate-1-¹³C (10 eq.), CD₂Cl₂ (0.6 mL), PEA (10 eq.), H₂O (10 μL);

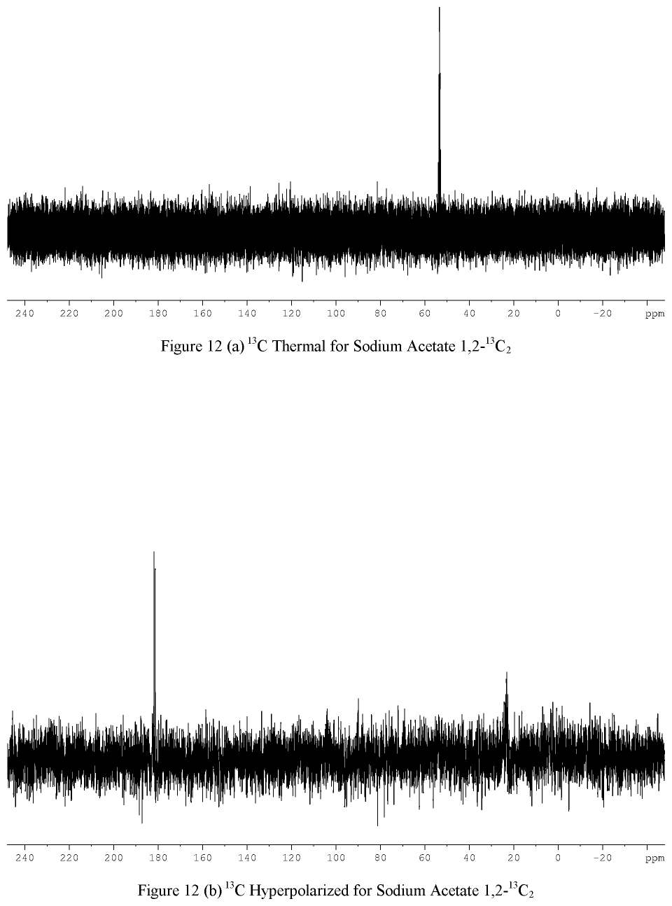

FIGS. 12 (a)-(b) are the spectra for (a) ¹³C thermal and (b) ¹³C hyperpolarised for Sodium Acetate 1,2-¹³C₂ using conditions: [IrCl(COD)(IMes)] (5 mM), sodium acetate 1,2-¹³C₂ (10 eq.), CD₂Cl₂ (0.6 mL), NH₃, H₂O (10 μL);

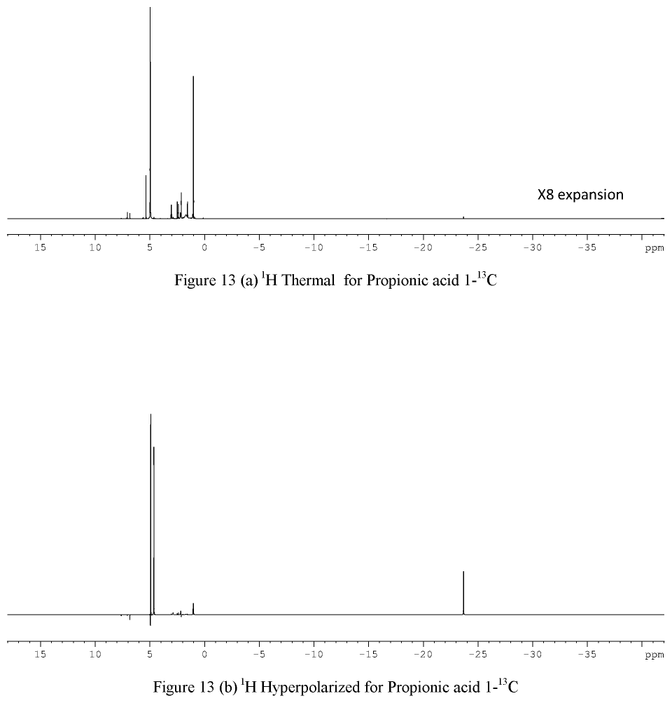

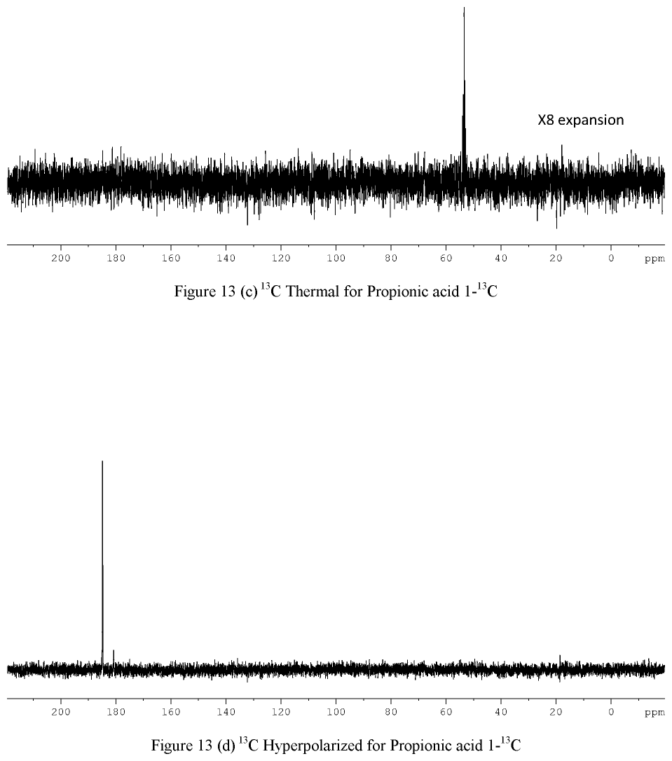

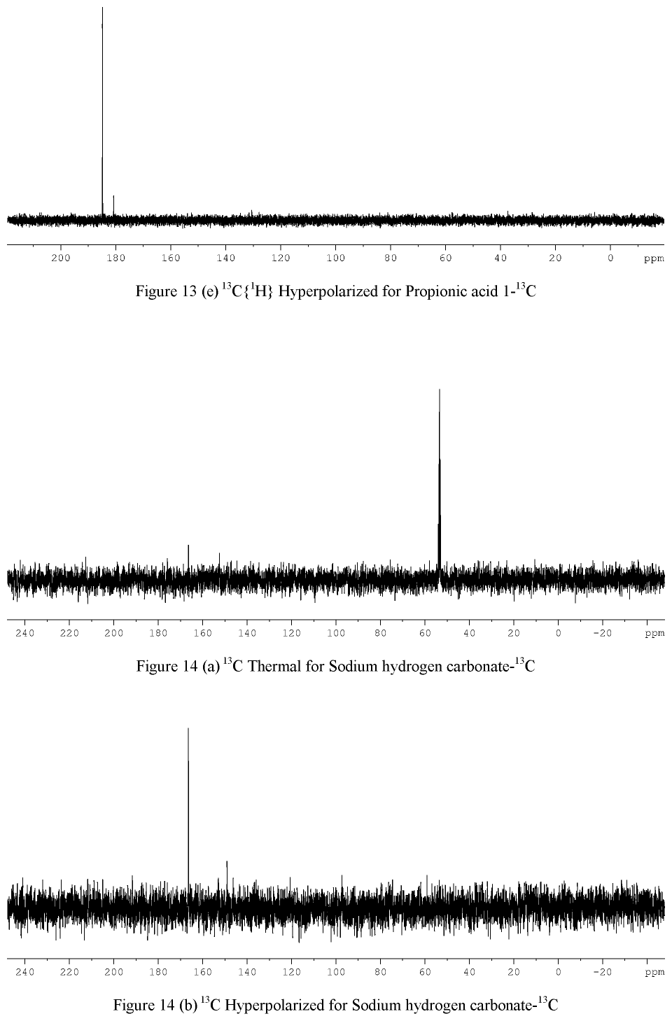

FIGS. 13 (a)-(e) are the spectra for (a) ¹H thermal, (b) ¹H hyperpolarised, (c) ¹³C thermal, (d) ¹³C hyperpolarised and (e) ¹³C hyperpolarised for Propionic acid 1-¹³C using conditions: [IrCl(COD)(IMes)] (5 mM), propionic acid 1-¹³C (10 eq.), CD₂Cl₂ (0.6 mL), NH₃, H₂O (10 μL), Cs₂CO₃ (10 eq.);

FIGS. 14 (a)-(b) are the spectra for (a) ¹³C thermal and (b) ¹³C hyperpolarised for sodium hydrogen carbonate-¹³C using conditions: [IrCl(COD)(IMes)] (5 mM), sodium hydrogen carbonate-¹³C (10 eq.), CD₂Cl₂ (0.6 mL), NH₃, H₂O (10 μL);

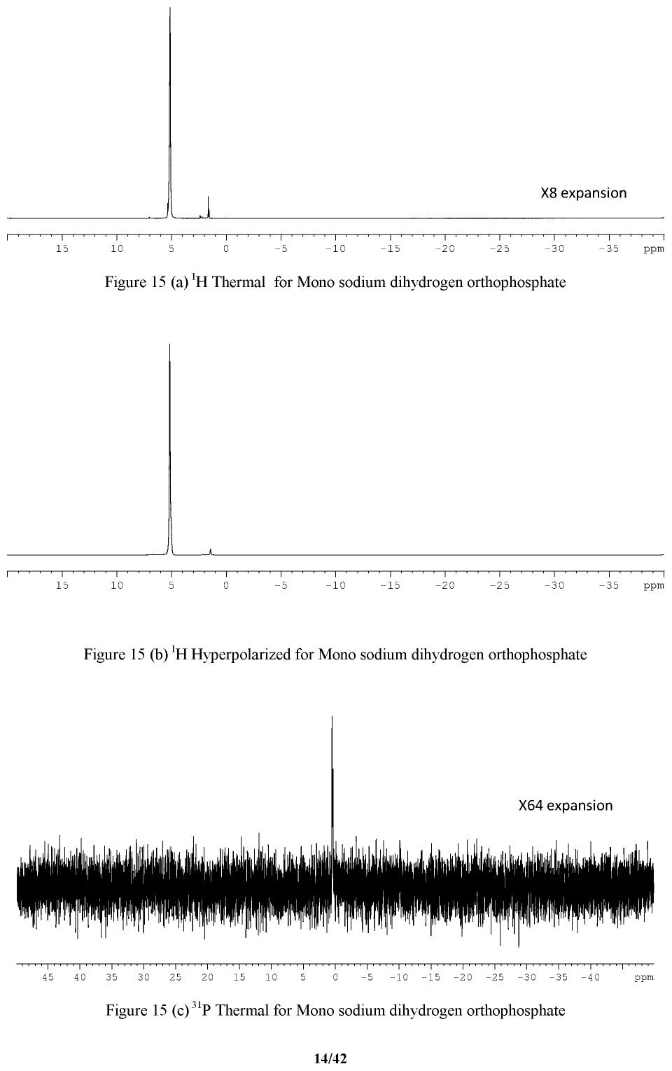

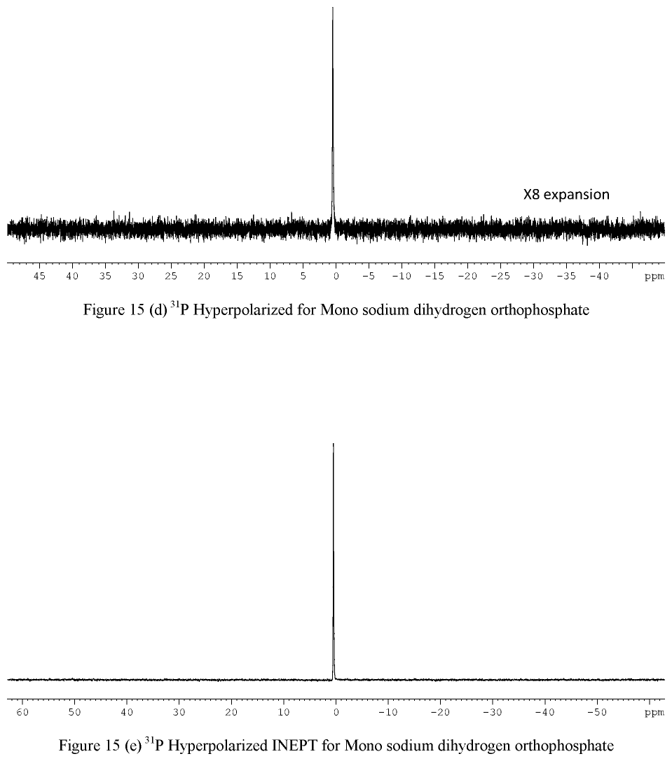

FIGS. 15 (a)-(e) are the spectra for (a) ¹H thermal, (b) ¹H hyperpolarised, (c) ³¹P thermal, (d) ³¹P hyperpolarised and (e) ³¹P hyperpolarised INEPT for Mono sodium dihydrogen orthophosphate using conditions: [IrCl(COD)(IMes)] (5 mM), mono sodium dihydrogen orthophosphate (10 eq.), CD₂Cl₂ (0.6 mL), NH₃, H₂O (10 μL);

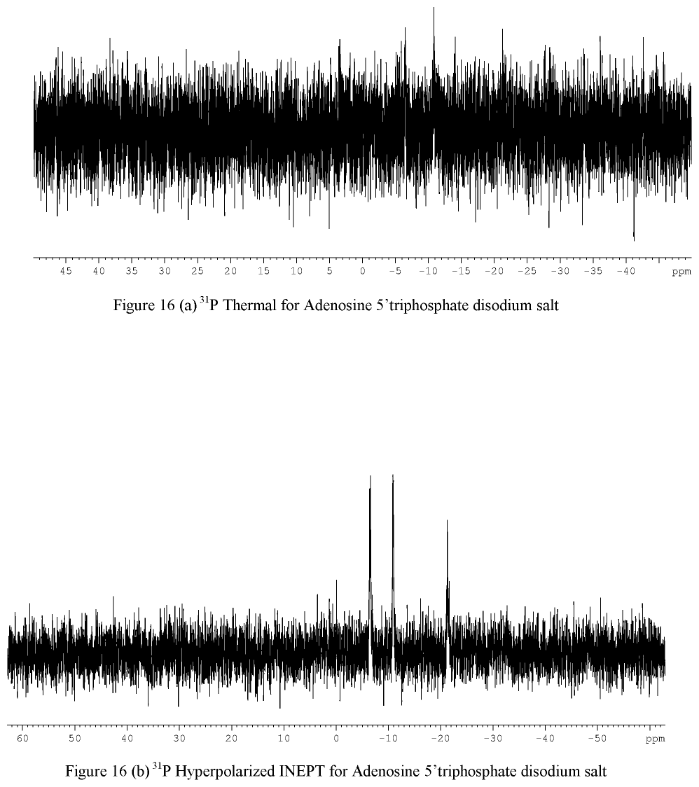

FIGS. 16 (a)-(b) are the spectra for (a) ³¹P thermal and (b) ³¹P hyperpolarised INEPT for Adenosine 5′triphosphate disodium salt using conditions: [IrCl(COD)(IMes)] (5 mM), Adenosine 5′triphosphate disodium salt (10 eq.), CD₂Cl₂ (0.6 mL), NH₃, H₂O (10 μL);

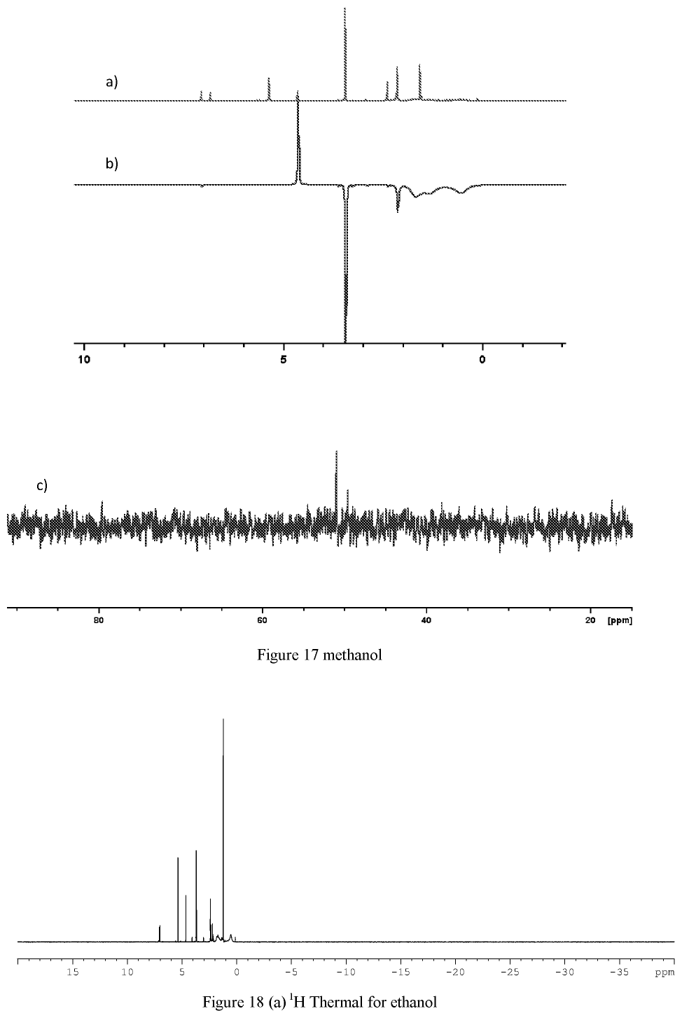

FIG. 17 is the spectra for methanol; using conditions: [IrCl(COD)(IMes)] (5 mM), methanol (1 μL), CD₂Cl₂ (0.6 mL), NH₃;

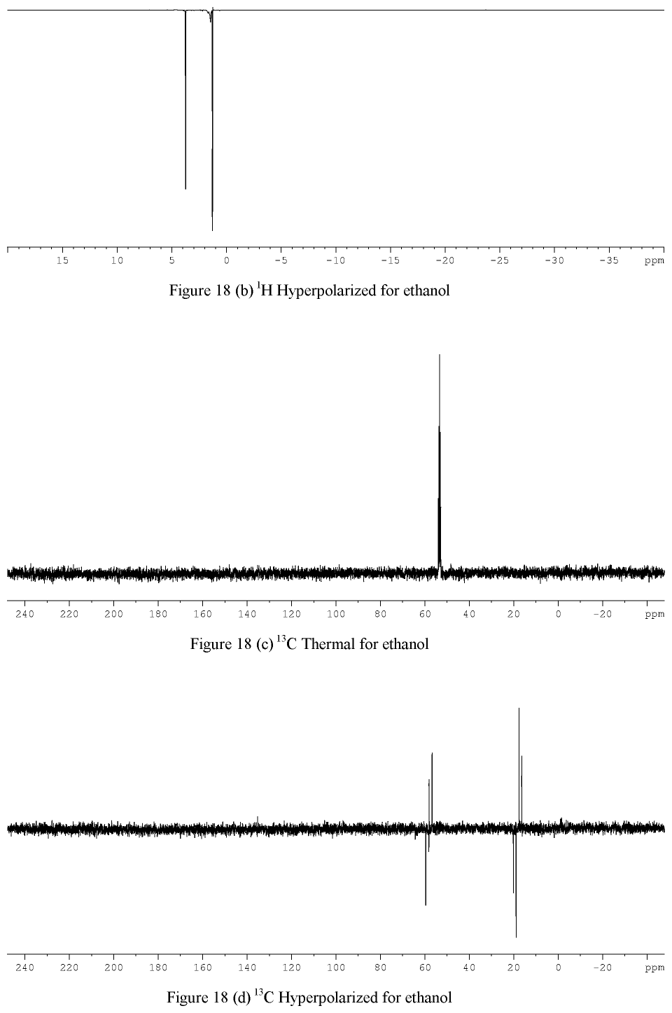

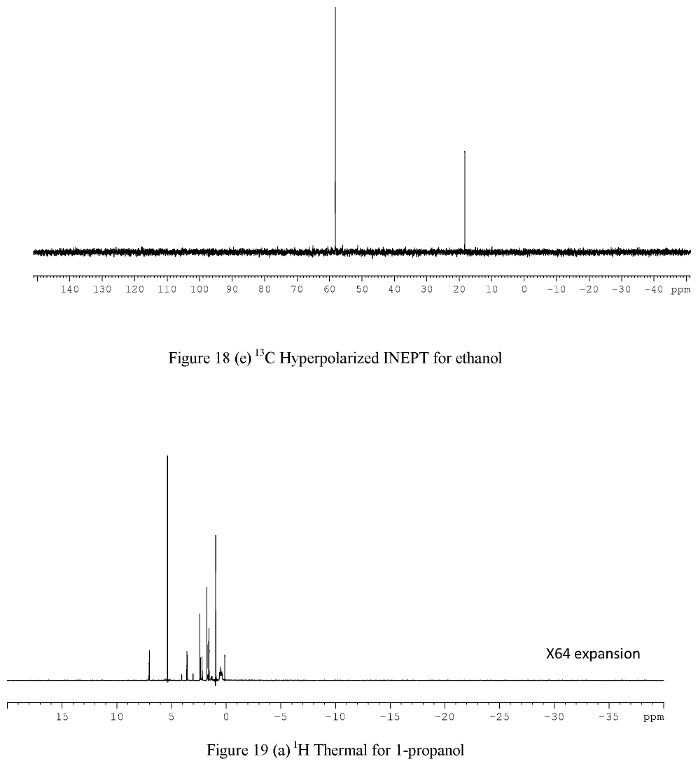

FIGS. 18 (a)-(e) are the spectra for (a) ¹H thermal, (b) ¹H hyperpolarised, (c) ³¹C thermal, (d) ³¹C hyperpolarised and (e) ¹³C hyperpolarised INEPT for ethanol using conditions: [IrCl(COD)(IMes)] (5 mM), ethanol (1 μL), CD₂Cl₂ (0.6 mL), NH₃;

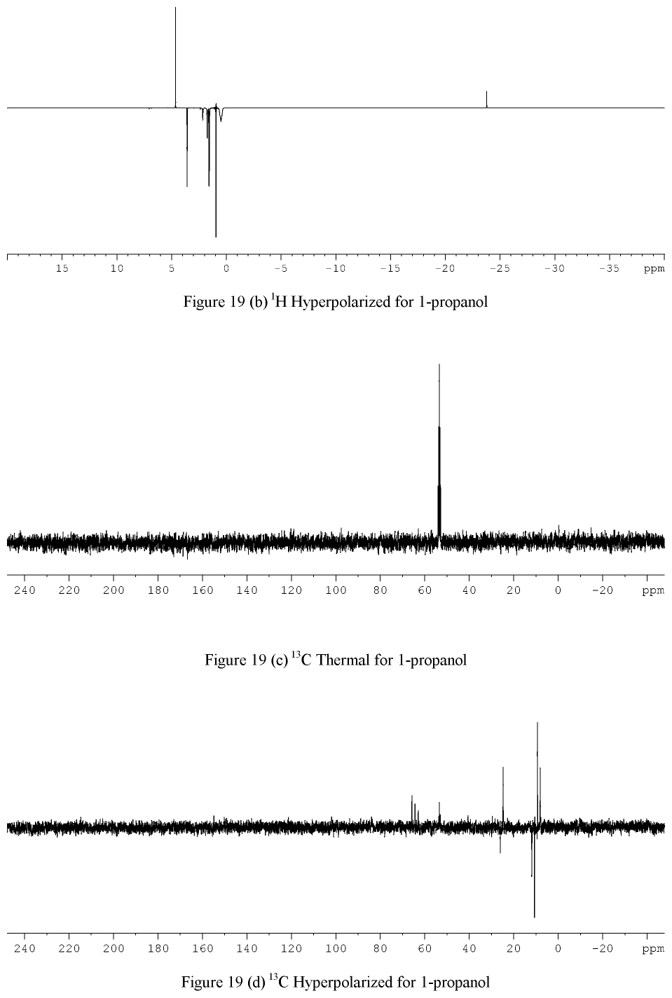

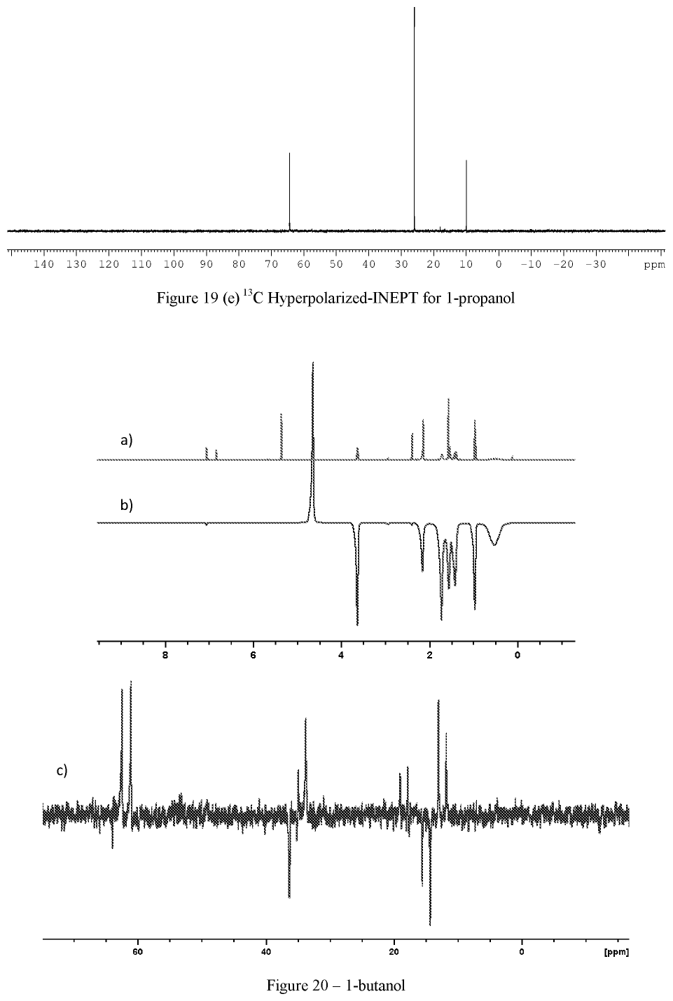

FIGS. 19 (a)-(e) are the spectra for (a) ¹H thermal, (b) ¹H hyperpolarised, (c) ³¹C thermal, (d) ³¹C hyperpolarised and (e) ¹³C hyperpolarised INEPT for 1-propanol using conditions: [IrCl(COD)(IMes)] (5 mM), 1-propanol (1 μL), CD₂Cl₂ (0.6 mL), NH₃;

FIGS. 20 (a)-(c) are the spectra for a) ¹H thermal ×32, b) ¹H SABRE hyperpolarisation (max enhancement 800 fold) and c) ¹³C SABRE hyperpolarisation for 1-butanol using conditions: [IrCl(COD)(IMes)] (5 mM), 1-butanol 1 (1 μL), CD₂Cl₂ (0.6 mL), NH₃;

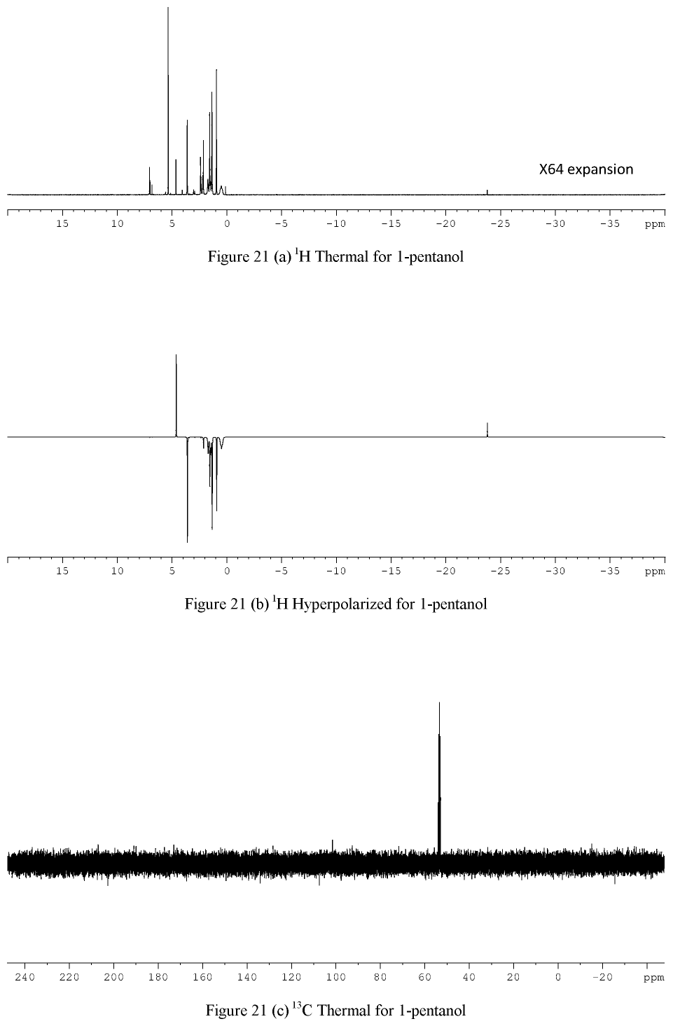

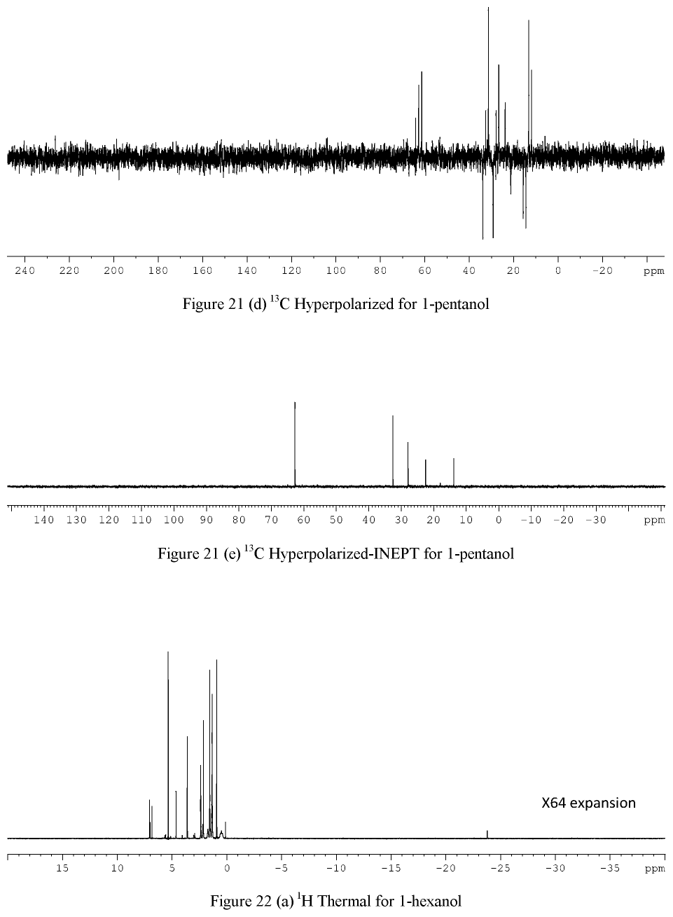

FIGS. 21 (a)-(e) are the spectra for (a) ¹H thermal, (b) ¹H hyperpolarised, (c) ³¹C thermal, (d) ³¹C hyperpolarised and (e) ¹³C hyperpolarised INEPT for 1-pentanol using conditions: [IrCl(COD)(IMes)] (5 mM), 1-pentanol (1 μL), CD₂Cl₂ (0.6 mL), NH₃;

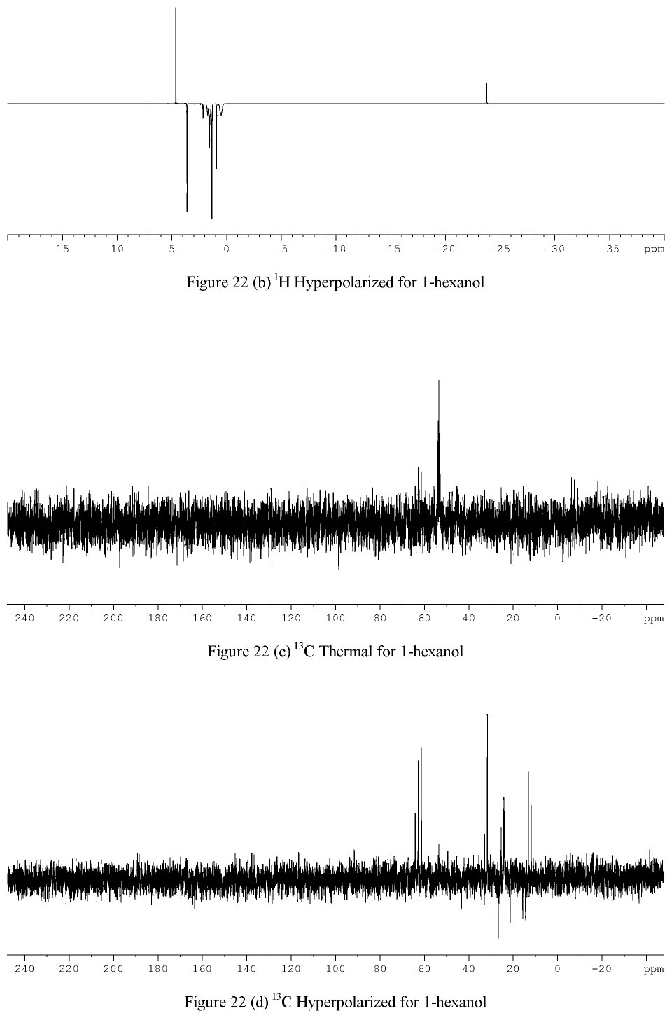

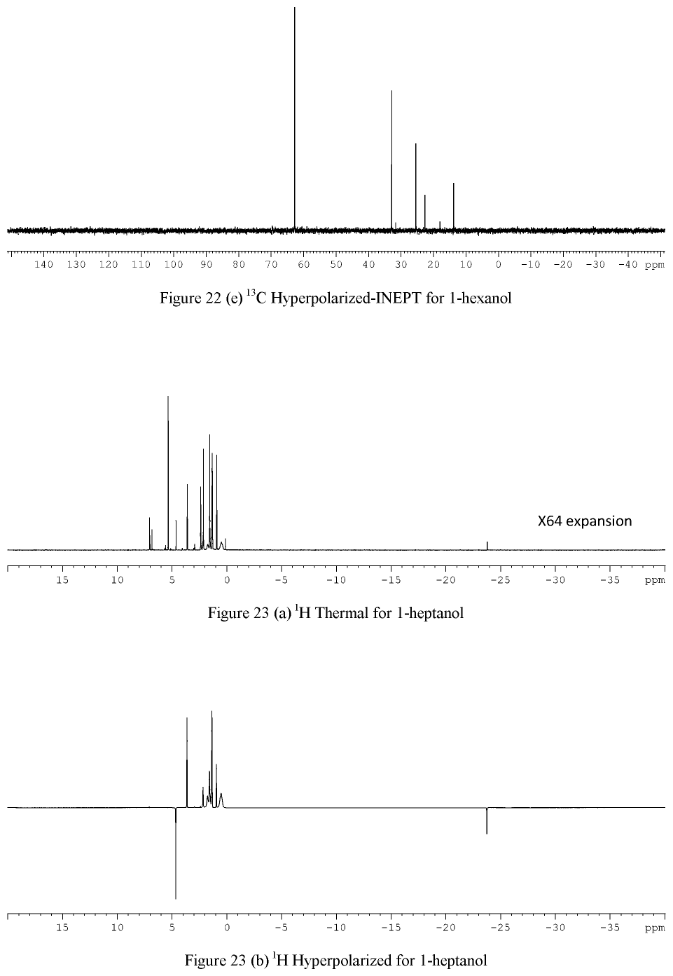

FIGS. 22 (a)-(e) are the spectra for (a) ¹H thermal, (b) ¹H hyperpolarised, (c) ³¹C thermal, (d) ³¹C hyperpolarised and (e) ¹³C hyperpolarised INEPT for 1-hexanol using conditions: [IrCl(COD)(IMes)] (5 mM), 1-hexanol (1 μL), CD₂Cl₂ (0.6 mL), NH₃;

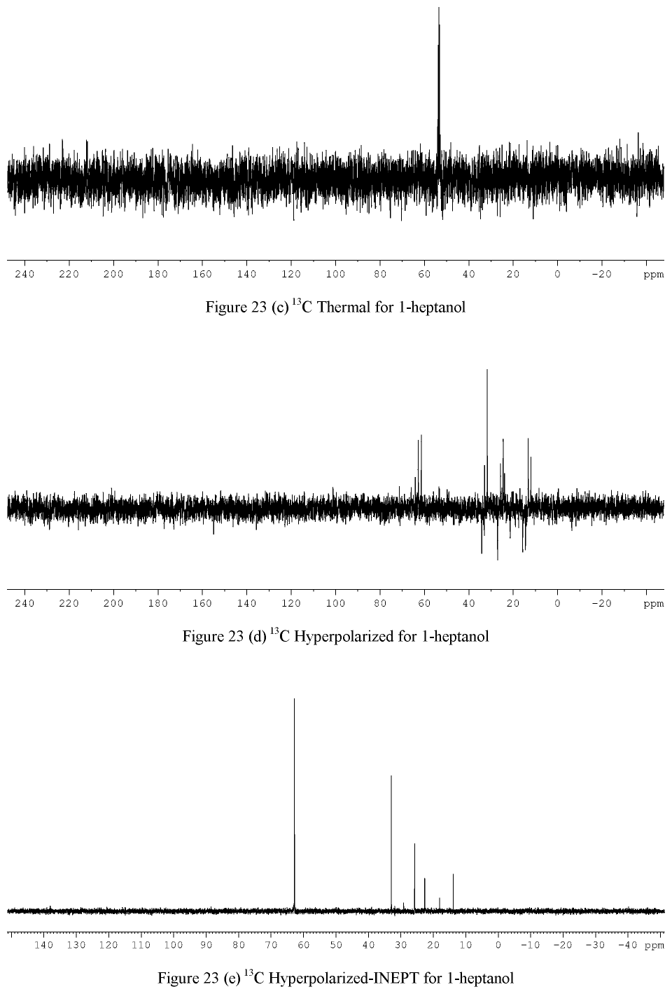

FIGS. 23 (a)-(e) are the spectra for (a) ¹H thermal, (b) ¹H hyperpolarised, (c) ³¹C thermal, (d) ³¹C hyperpolarised and (e) ¹³C hyperpolarised INEPT for 1-heptanol using conditions: [IrCl(COD)(IMes)] (5 mM), 1-heptanol (1 μL), CD₂Cl₂ (0.6 mL), NH₃;

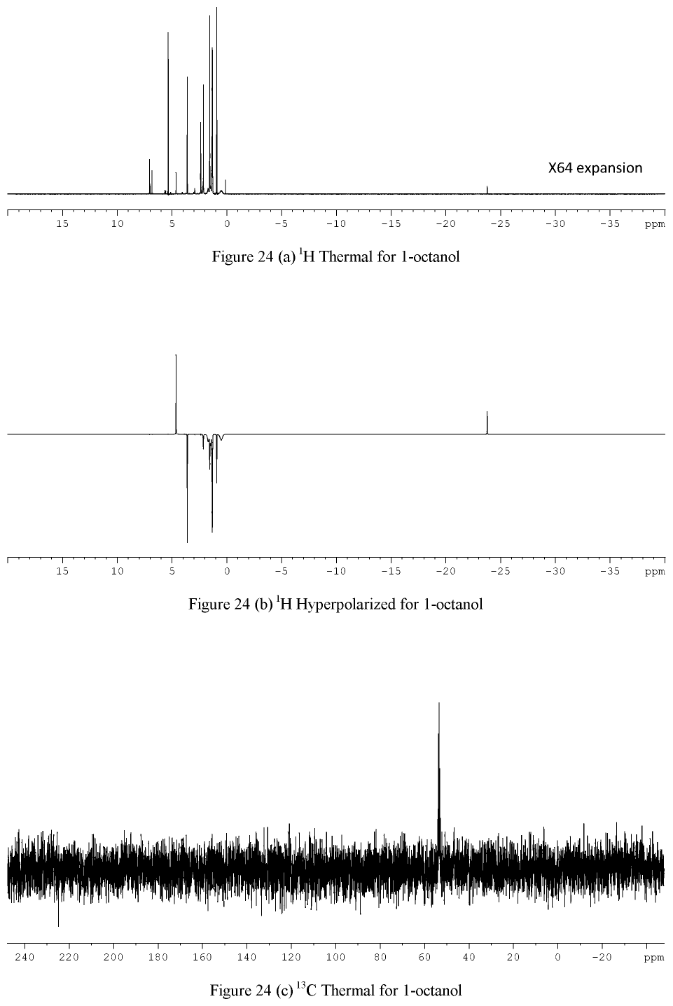

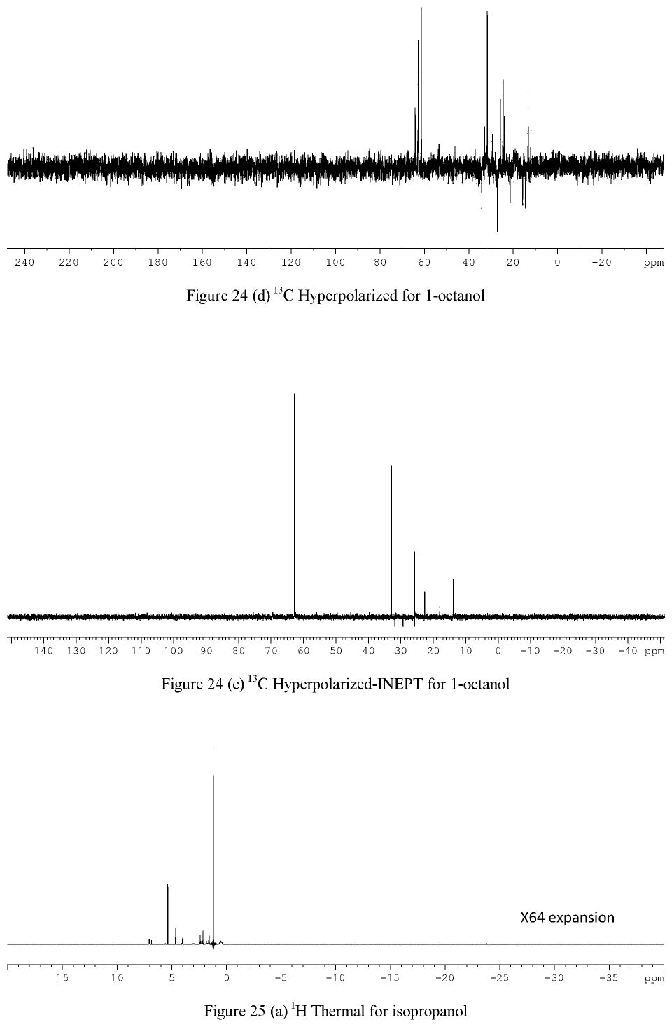

FIGS. 24 (a)-(e) are the spectra for (a) ¹H thermal, (b) ¹H hyperpolarised, (c) ³¹C thermal, (d) ³¹C hyperpolarised and (e) ¹³C hyperpolarised INEPT for 1-octanol using conditions: [IrCl(COD)(IMes)] (5 mM), 1-octanol (1 μL), CD₂Cl₂ (0.6 mL), NH₃;

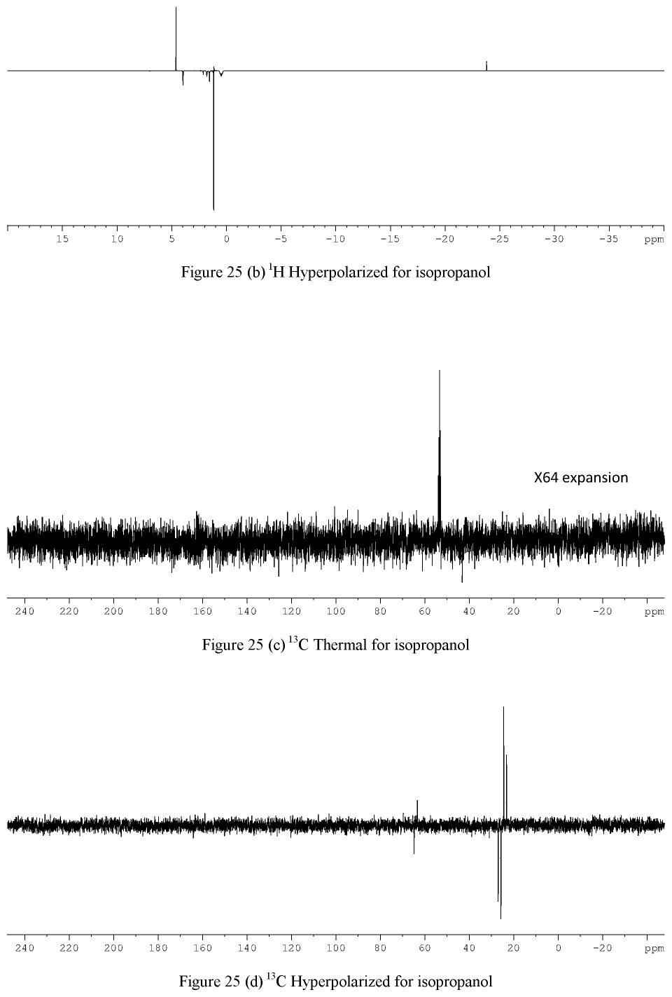

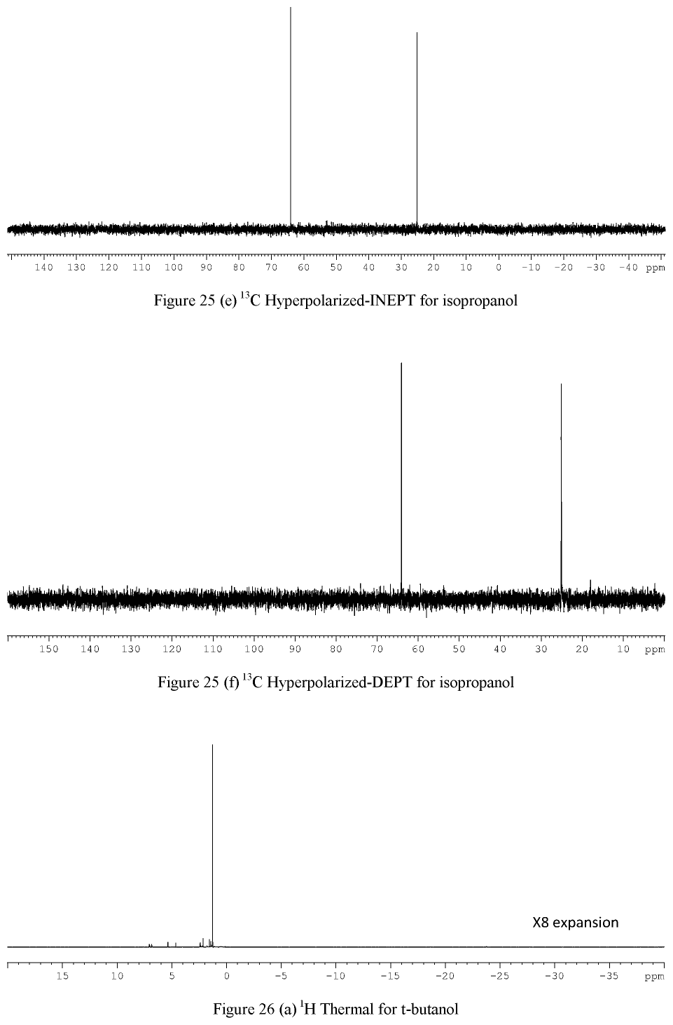

FIGS. 25 (a)-(f) are the spectra for (a) ¹H thermal, (b) ¹H hyperpolarised, (c) ³¹C thermal, (d) ³¹C hyperpolarised, (e) ¹³C hyperpolarised INEPT and (f) ¹³C hyperpolarised-DEPT for isopropanol using conditions: [IrCl(COD)(IMes)] (5 mM), isopropanol (1 μL), CD₂Cl₂ (0.6 mL), NH₃;

FIGS. 26 (a)-(c) are the spectra for (a) ¹H thermal, (b) ¹H hyperpolarised and (c) ³¹C thermal for t-butanol using conditions: [IrCl(COD)(IMes)] (5 mM), tert-butanol (1 μL), CD₂Cl₂ (0.6 mL), NH₃;

FIGS. 27 (a)-(c) are the spectra for a 10 fold excess of glucose: a) ¹H thermal ×4, b) ¹H SABRE hyperpolarisation (max enhancement 40 fold) for D-glucose-¹³C₆;

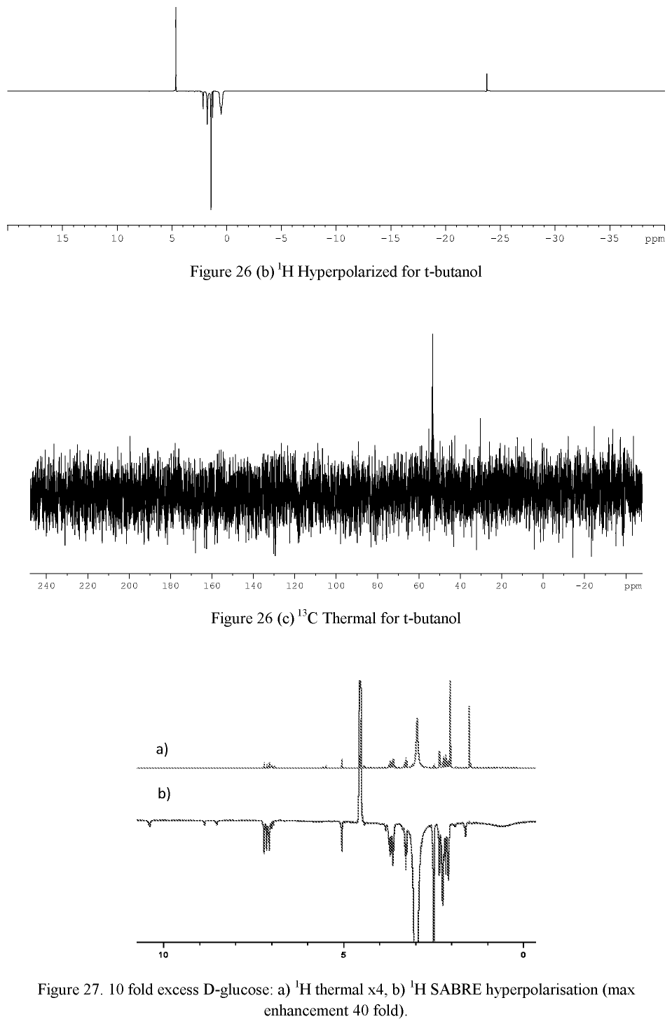

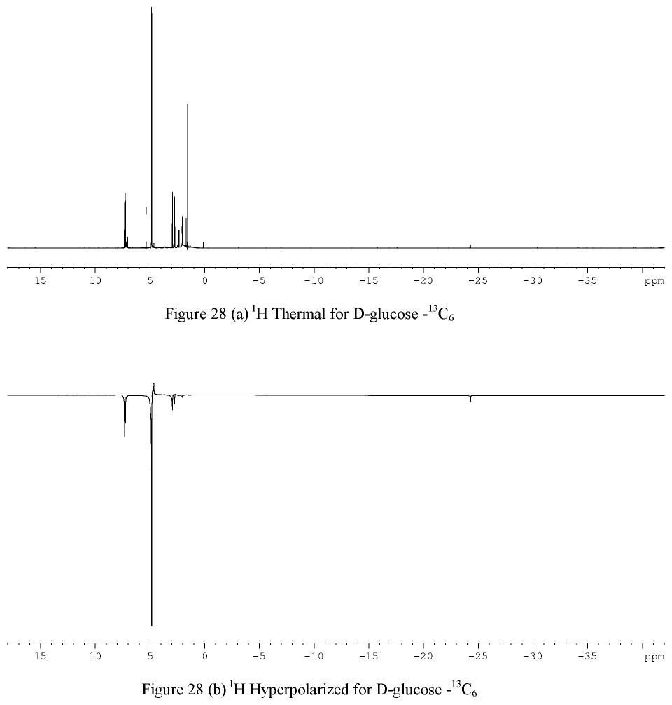

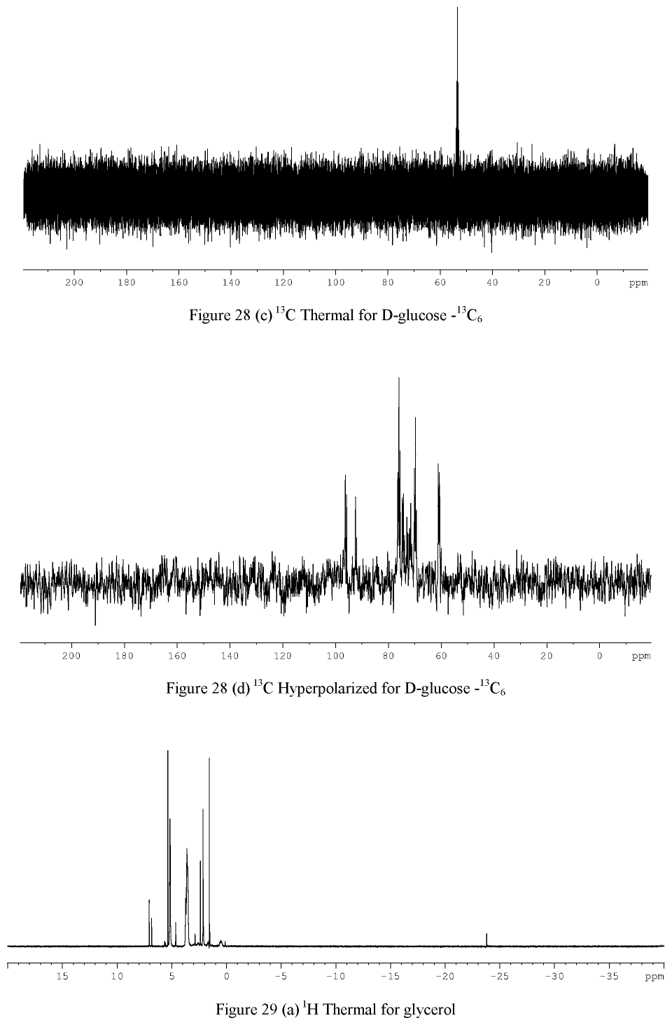

FIGS. 28 (a)-(d) are the spectra for (a) ¹H thermal, (b) ¹H hyperpolarised, (c) ¹³C thermal and (d) ¹³C hyperpolarised for D-glucose-¹³C₆ using conditions: [IrCl(COD)(1,3-bis(2,4,6-trimethylphenyl)-4,5-dimethylimidazole)] (5 mM), glucose-¹³C₆ (10 eq.), CD₂Cl₂ (0.6 mL), PEA (1 μL), H₂O (5 μL);

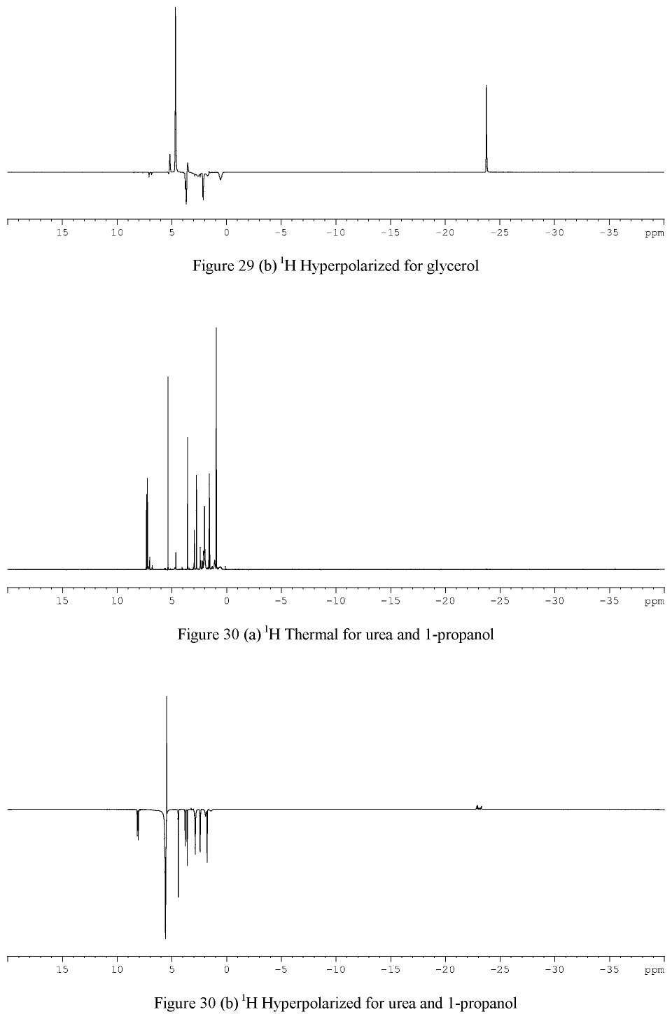

FIGS. 29 (a)-(b) are the spectra for (a) ¹H thermal and (b) ¹H hyperpolarised for glycerol using conditions: [IrCl(COD)(IMes)] (5 mM), glycerol (20 eq.), CD₂Cl₂ (0.6 mL), NH₃;

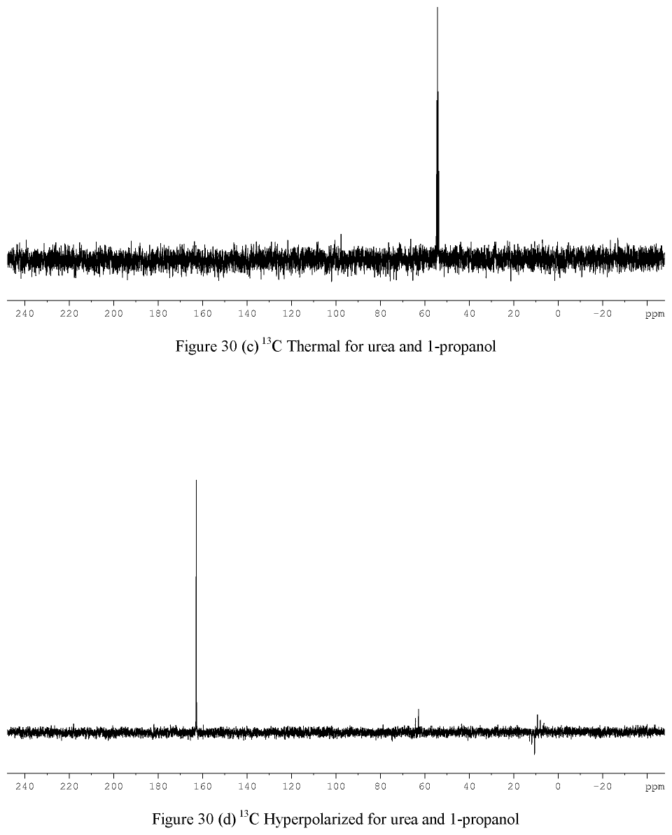

FIGS. 30 (a)-(d) are the spectra for (a) ¹H thermal, (b) ¹H hyperpolarised, (c) ¹³C thermal and (d) ¹³C hyperpolarised for a mixture of uea and 1-propanol using conditions: [IrCl(COD)(IMes)] (5 mM), Urea-1-¹³C (10 eq.), 1-propanol (1 μL), PEA (1 μL CD₂Cl₂ (0.6 mL), H₂O (5 μL), NH₃; FIGS. 31 (a)-(c) are the spectra for n-butanol: a) ¹H thermal ×32, b) ¹H SABRE hyperpolarisation (max enhancement 800 fold) and c) ¹³C SABRE hyperpolarisation;

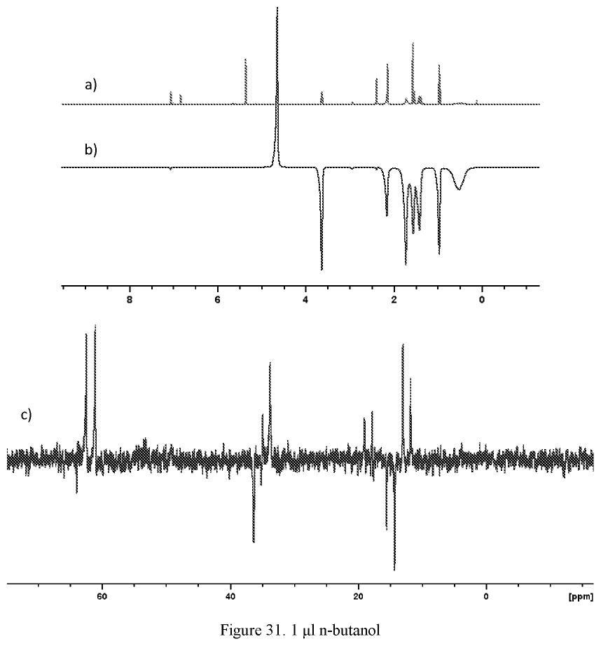

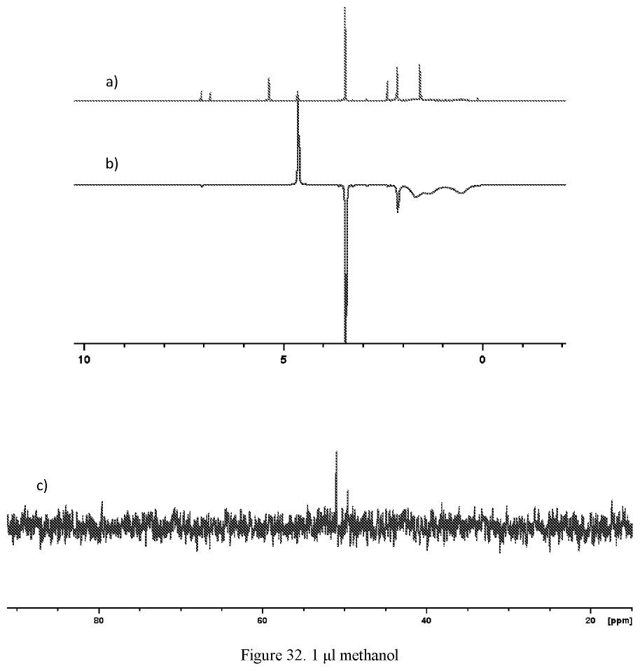

FIGS. 32 (a)-(c) are the spectra for 1 μl methanol: a) ¹H thermal ×64, b) ¹H SABRE hyperpolarisation (max enhancement 900 fold) and c) ¹³C SABRE hyperpolarisation;

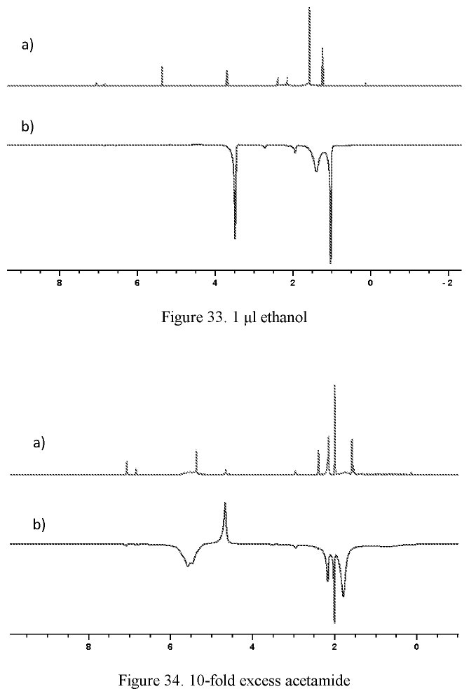

FIGS. 33 (a)-(b) are the spectra for ethanol: 1 μl ethanol: a) ¹H thermal, b) ¹H SABRE hyperpolarisation (max enhancement 50-fold low because of the NH₃ amount);

FIGS. 34 (a)-(b) are the spectra for acetamide: 10-fold excess acetamide: a) ¹H thermal, b) ¹H SABRE hyperpolarisation (max enhancement 400 fold);

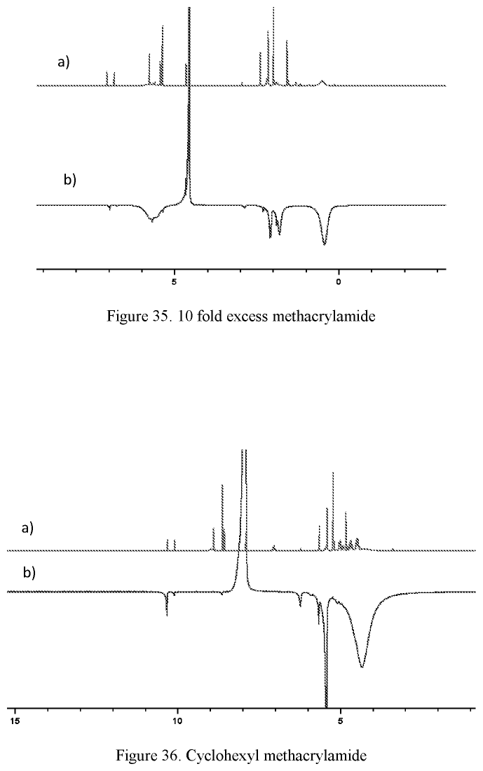

FIGS. 35 (a)-(b) are the spectra for Methacrylamide: 10 fold excess methacrylamide: a) ¹H thermal ×16, b) ¹H SABRE hyperpolarisation (max enhancement 200 fold);

FIGS. 36 (a)-(b) are the spectra for Cyclohexyl methacrylamide 10-fold excess: a) ¹H thermal, b) ¹H SABRE hyperpolarisation (max enhancement 150 fold);

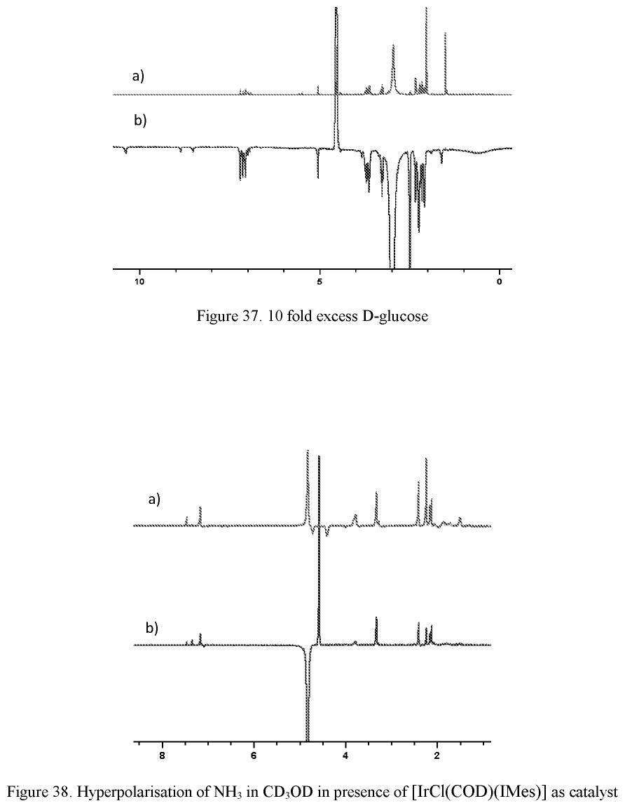

FIGS. 37 (a)-(b) are the spectra for 10 fold excess D-glucose: a) ¹H thermal ×4, b) ¹H SABRE hyperpolarisation (max enhancement 40 fold);

FIGS. 38 (a)-(b) are the spectra for NH₃ in CD₃OD: a) ¹H spectrum after first shake under p-H₂ (start of the activation process), b) after 1 min of the activation, the methanol does hyperpolarisation after H/D exchange;

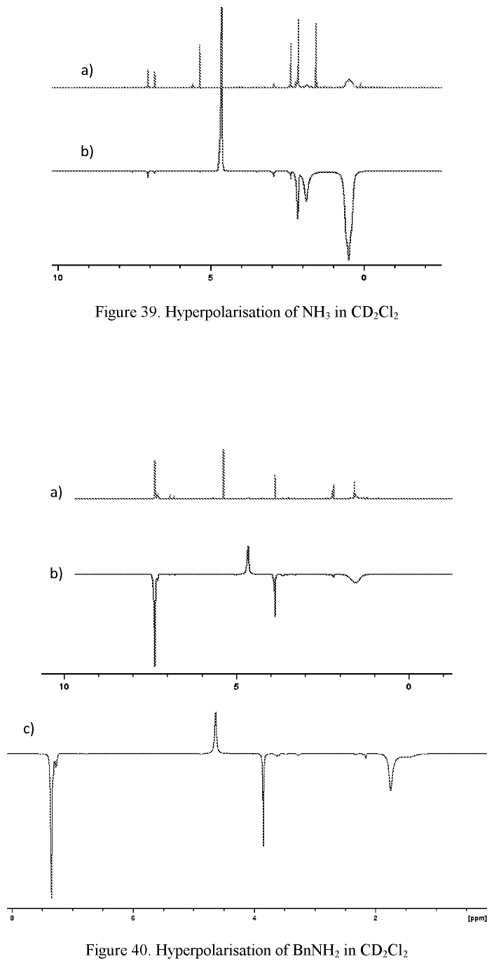

FIGS. 39 (a)-(b) are the spectra for hyperpolarisation of NH₃ in CD₂Cl₂ in presence of 1 (IMesIrClCOD) as catalyst a) ¹H thermal ×16, b) ¹H SABRE hyperpolarisation (max enhancement 94 fold);

FIGS. 40 (a)-(c) are the spectra for hyperpolarisation of benzylamine in CD₂Cl₂ in presence of (IMesIrClCOD) as catalyst a) ¹H thermal ×8, b) ¹H SABRE hyperpolarisation (max enhancement 160 fold) and c) after adding 1 μl of D₂O (max enhancement of H₂O 215 fold); and

FIGS. 41 (a)-(c) are the spectra for Hyperpolarisation of ¹⁵BnNH₂ in CD₂Cl₂ in presence of (IMesIrClCOD) as catalyst a) ¹H thermal ×4, b) ¹H SABRE hyperpolarisation (max enhancement 60 fold) and c) ¹⁵N hyperpolarisation.

### REFERENCES

1. C. R. Bowers, D. P. Weitekamp, Journal of the American Chemical Society 109, 5541-5542 (1987).
2. J. Natterer, J. Bargon, Progress in Nuclear Magnetic Resonance Spectroscopy 31, 293-315 (1997).
3. R. A. Green, R. W. Adams, S. B. Duckett, R. E. Mewis, D. C. Williamson, G. G. R. Green, Progress in Nuclear Magnetic Resonance Spectroscopy 67, 1-48 (2012).
4. R. W. Adams, J. A. Aguilar, K. D. Atkinson, M. J. Cowley, P. I. P. Elliott, S. B. Duckett, G. G. R. Green, I. G. Khazal, J. Lopez-Serrano, D. C. Williamson, Science 323, 1708-1711 (2009).
5. P. J. Rayner, M. J. Burns, A. M. Olaru, P. Norcott, M. Fekete, G. G. R. Green, L. A. R. Highton, R. E. Mewis, S. B. Duckett, Proceedings of the National Academy of Sciences of the United States of America 114, E3188-E3194 (2017).
6. H. F. Zeng, J. D. Xu, J. Gillen, M. T. McMahon, D. Artemov, J. M. Tyburn, J. A. B. Lohman, R. E. Mewis, K. D. Atkinson, G. G. R. Green, S. B. Duckett, P. C. M. van Zijl, Journal ofMagnetic Resonance 237, 73-78 (2013).
7. E. B. Ducker, L. T. Kuhn, K. Munnemann, C Griesinger, Journal of Magnetic Resonance 214, 159-165 (2012).
8. R. E. Mewis, R. A. Green, M. C. R. Cockett, M. J. Cowley, S. B. Duckett, G. G. R. Green, R. O. John, P. J. Rayner, D. C. Williamson, Journal of Physical Chemistry B 119, 1416-1424 (2015).
9. R. E. Mewis, K. D. Atkinson, M. J. Cowley, S. B. Duckett, G. G. R. Green, R. A. Green, L. A. R. Highton, D. Kilgour, L. S. Lloyd, J. A. B. Lohman, D. C. Williamson, Magnetic Resonance in Chemistry 52, 358-369 (2014).
10. J. F. P. Colell, A. W. J. Logan, Z. J. Zhou, R. V. Shchepin, D. A. Barskiy, G. X. Ortiz, Q. Wang, S. J. Malcolmson, E. Y. Chekmenev, W. S. Warren, T. Theis, Journal of Physical Chemistry C 121, 6626-6634 (2017).
11. D. A. Barskiy, R. V. Shchepin, C. P. N. Tanner, J. F. P. Colell, B. M. Goodson, T. Theis, W. S. Warren, E. Y. Chekmenev, Chemphyschem 18, 1493-1498 (2017).
12. V. V. Zhivonitko, I. V. Skovpin, I. V. Koptyug, Chemical Communications 51, 2506-2509 (2015).
13. M. J. Burns, P. J. Rayner, G. G. R. Green, L. A. R. Highton, R. E. Mewis, S. B. Duckett, Journal of Physical Chemistry B 119, 5020-5027 (2015).
14. R. W. Adams, S. B. Duckett, R. A. Green, D. C. Williamson, G. G. R. Green, Journal of Chemical Physics 131, (2009).
15. A. N. Pravdivtsev, A. V. Yurkovskaya, H. M. Vieth, K. L. Ivanov, R. Kaptein, Chemphyschem 14, 3327-3331 (2013).
16. A. N. Pravdivtsev, A. V. Yurkovskaya, K. L. Ivanov, H. M. Vieth, Journal of Magnetic Resonance 254, 35-47 (2015).
17. N. Eshuis, R. Aspers, B. J. A. van Weerdenburg, M. C. Feiters, F. Rutjes, S. S. Wijmenga, M. Tessari, Journal of Magnetic Resonance 265, 59-66 (2016).
18. K. D. Atkinson, M. J. Cowley, P. I. P. Elliott, S. B. Duckett, G. G. R. Green, J. Lopez-Serrano, A. C. Whitwood, Journal of the American Chemical Society 131, 13362-13368 (2009).
19. K. D. Atkinson, M. J. Cowley, S. B. Duckett, P. I. P. Elliott, G. G. R. Green, J. Lopez-Serrano, I. G. Khazal, A. C. Whitwood, Inorganic Chemistry 48, 663-670 (2009).
20. T. Theis, M. Truong, A. M. Coffey, E. Y. Chekmenev, W. S. Warren, Journal of Magnetic Resonance 248, 23-26 (2014).
21. M. Carravetta, 0. G. Johannessen, M. H. Levitt, Physical Review Letters 92, (2004).
22. T. Theis, G. X. Ortiz, A. W. J. Logan, K. E. Claytor, Y. Feng, W. P. Huhn, V. Blum, S. J. Malcolmson, E. Y. Chekmenev, Q. Wang, W. S. Warren, Science Advances 2, (2016).
23. A. M. Olaru, S. S. Roy, L. S. Lloyd, S. Coombes, G. G. R. Green, S. B. Duckett, Chemical Communications 52, 7842-7845 (2016).
24. S. S. Roy, P. Norcott, P. J. Rayner, G. G. R. Green, S. B. Duckett, Angewandte Chemie International Edition 55, 15642-15645 (2016).
25. S. S. Roy, P. J. Rayner, P. Norcott, G. G. R. Green, S. B. Duckett, Physical Chemistry Chemical Physics 18, 24905-24911 (2016).
26. M. J. Cowley, R. W. Adams, K. D. Atkinson, M. C. R. Cockett, S. B. Duckett, G. G. R. Green, J. A. B. Lohman, R. Kerssebaum, D. Kilgour, R. E. Mewis, Journal of the American Chemical Society 133, 6134-6137 (2011).
27. O. G. Salnikov, K. V. Kovtunov, D. A. Barskiy, A. K. Khudorozhkov, E. A. Inozemtseva, I. P. Prosvirin, V. I. Bukhtiyarov, I. V. Koptyug, Acs Catalysis 4, 2022-2028 (2014).
28. M. Leutzsch, L. M. Wolf, P. Gupta, M. Fuchs, W. Thiel, C. Fares, A. Furstner, Angewandte Chemie-International Edition 54, 12431-12436 (2015).
29. R. V. Shchepin, D. A. Barskiy, A. M. Coffey, B. M. Goodson, E. Y. Chekmenev, Chemistryselect 1, 2552-2555 (2016).
30. C. Godard, S. B. Duckett, S. Polas, R. Tooze, A. C. Whitwood, Dalton Transactions, 2496-2509 (2009).
31. D. J. Fox, S. B. Duckett, C. Flaschenriem, W. W. Brennessel, J. Schneider, A. Gunay, R. Eisenberg, Inorganic Chemistry 45, 7197-7209 (2006).
32. D. Blazina, S. B. Duckett, P. J. Dyson, J. A. B. Lohman, Angewandte Chemie-International Edition 40, 3874-+(2001).
33. S. A. Colebrooke, S. B. Duckett, J. A. B. Lohman, R. Eisenberg, Chemistry-a European Journal 10, 2459-2474 (2004).
34. J. S. M. Samec, J. E. Backvall, P. G. Andersson, P. Brandt, Chem. Soc. Rev. 35, 237-248 (2006).
35. S. E. Clapham, A. Hadzovic, R. H. Morris, Coordination Chemistry Reviews 248, 2201-2237 (2004).
36. M. Patel, R. K. Saunthwal, A. K. Verma, Accounts of Chemical Research 50, 240-254 (2017).
37. J. S. Anderson, J. Rittle, J. C. Peters, Nature 501, 84-+(2013).
38. S. S. Roy, P. Norcott, P. J. Rayner, G. G. R. Green, S. B. Duckett, Chem. Eur. J. (in press) doi: 10.1002/chem.201702767 (2017).
39. Lehmkuhl et al “Hyperpolarising Water with Parahydrogen”, ChemPhysChem 10.1002/cphc.201700750

1. A method for the preparation of a hyperpolarised target molecule, wherein said molecule comprises at least one —OH, —NH or —SH moiety, via proton exchange from a polarisable molecule, said method comprising the steps of:
(i) preparing a fluid containing a transfer catalyst; parahydrogen; and a polarisable molecule containing at least one exchangeable proton, such as, an —OH, —NH or —SH moiety;
(ii) applying a magnetic field or radio frequency excitation such that hyperpolarisation is transferred from parahydrogen to the polarisable molecule when bound to the magnetisation transfer catalyst;
(iii) separately or simultaneously introducing a target molecule, wherein said target molecule contains at least one —OH, —NH or —SH exchangeable proton, enabling hyperpolarisation transfer via proton exchange with the polarisable molecule.

2. A method according to claim 1 wherein the target molecule comprises a non-hydrogenatable hydrocarbon moiety and the hyperpolarisation of the target molecule occurs by the proton exchange RELAY effect.

3.-5. (canceled)

6. A method according to claim 1 wherein the hyperpolarisation is achieved by polarisation transfer through spin refrigeration, DNP, para-hydrogen induced polarisation (PHIP), SABRE or from a suitable molecule in a singlet state.

7.-13. (canceled)

14. A method according to claim 1 wherein the magnetic field is an ultra-low magnetic field which is <<1 G (<10⁻⁶ T).

15.-20. (canceled)

21. A method according to claim 1 wherein the polarisable molecule contains at least one —SH moiety.

22. A method according to claim 21 wherein the polarisable molecule comprises a thiol or thioamide moiety.

23.-30. (canceled)

31. A method according to claim 1 wherein the target molecule contains at least one —SH moiety.

32. A method according to claim 31 wherein the target molecule comprises a thiol or thioamide moiety.

33. A method according to claim 31 wherein the target molecule comprises:
(i) HSR wherein R represents H, alkyl_{C1-20}, aryl, vinyls, or any combination thereof; and
(ii) thioamides, thioacids, thioureas and xanthates.

34. A method according to claim 1 wherein the hyperpolarisation transfer catalyst comprises a metal atom selected from the group consisting of Ru, Rh, Ir, W, Pd and Pt.

35. (canceled)

36. A method according to claim 34 wherein the hyperpolarisation transfer catalyst comprises a metal atom is iridium with at least one N-heterocyclic carbene (NHC) ligand.

37. A method according to claim 36 wherein the N-heterocyclic carbene (NHC) ligand is selected from:

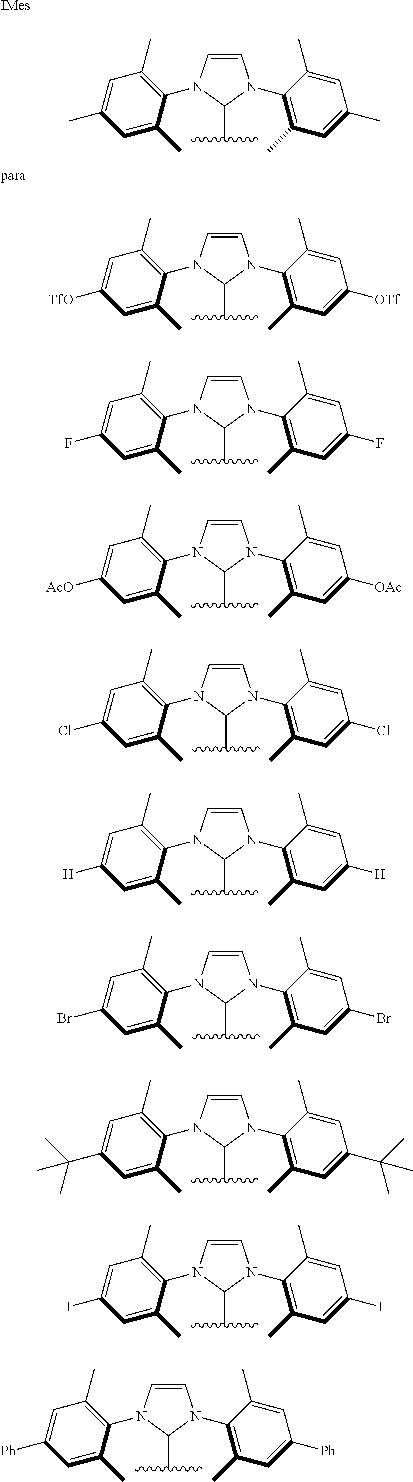

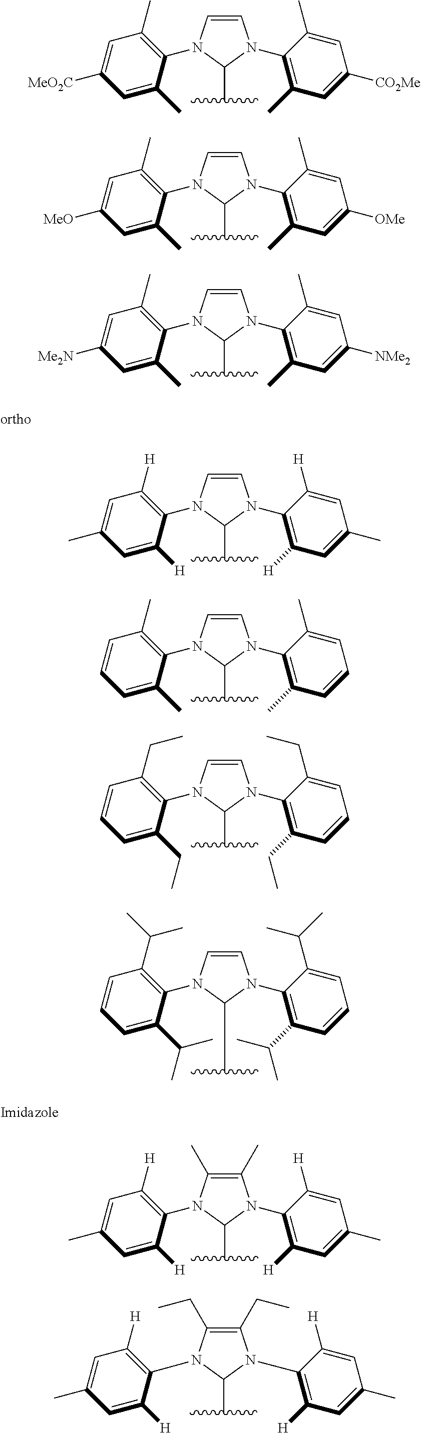

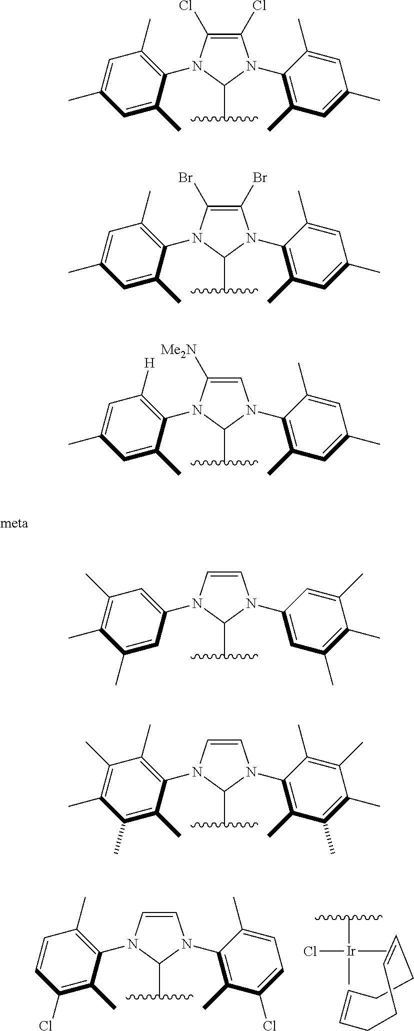

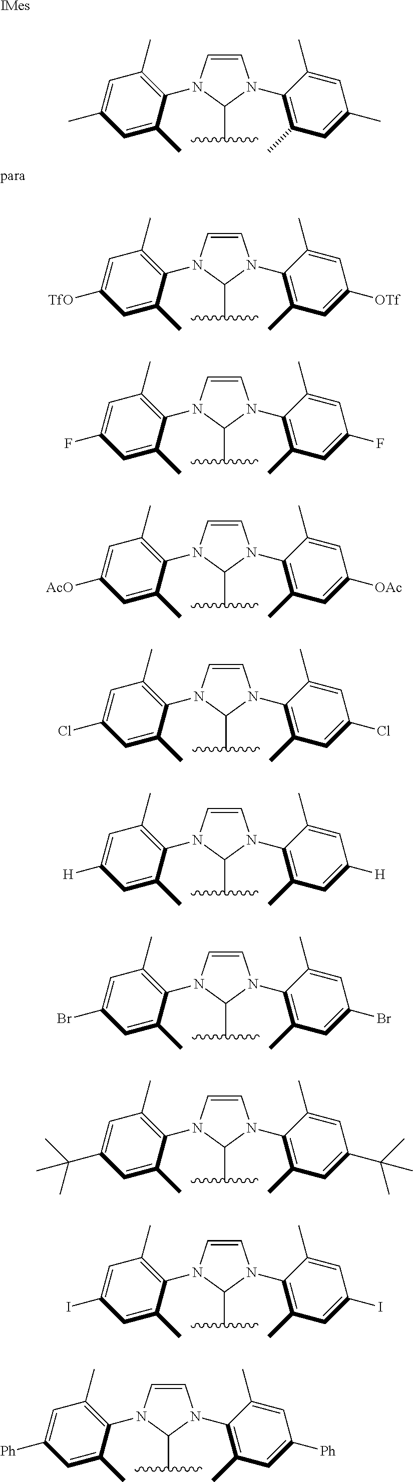

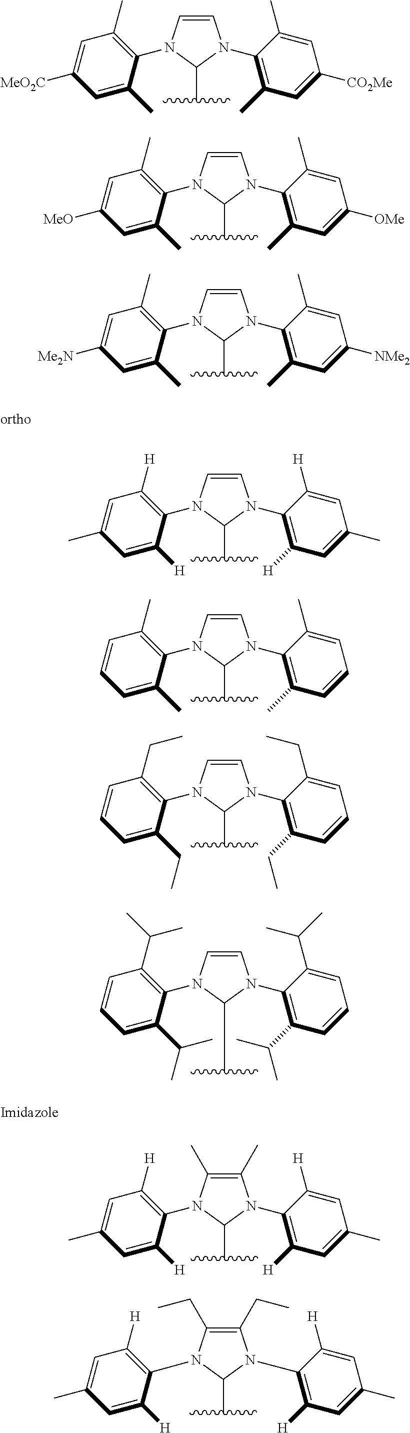

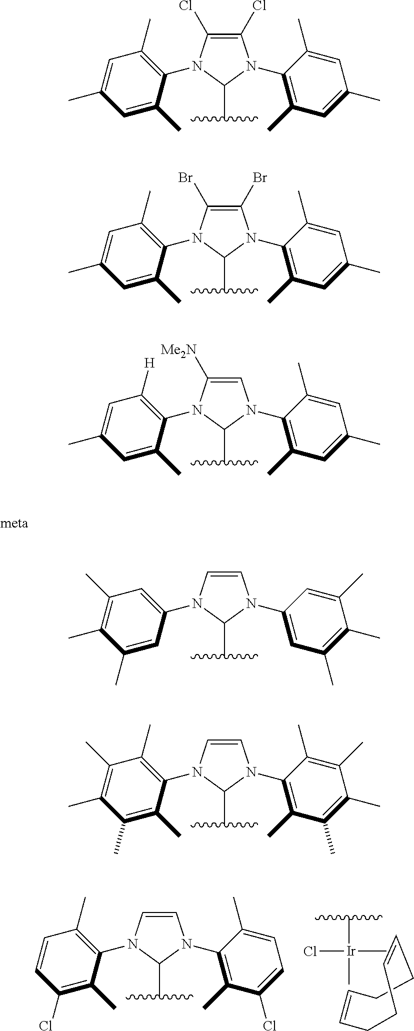

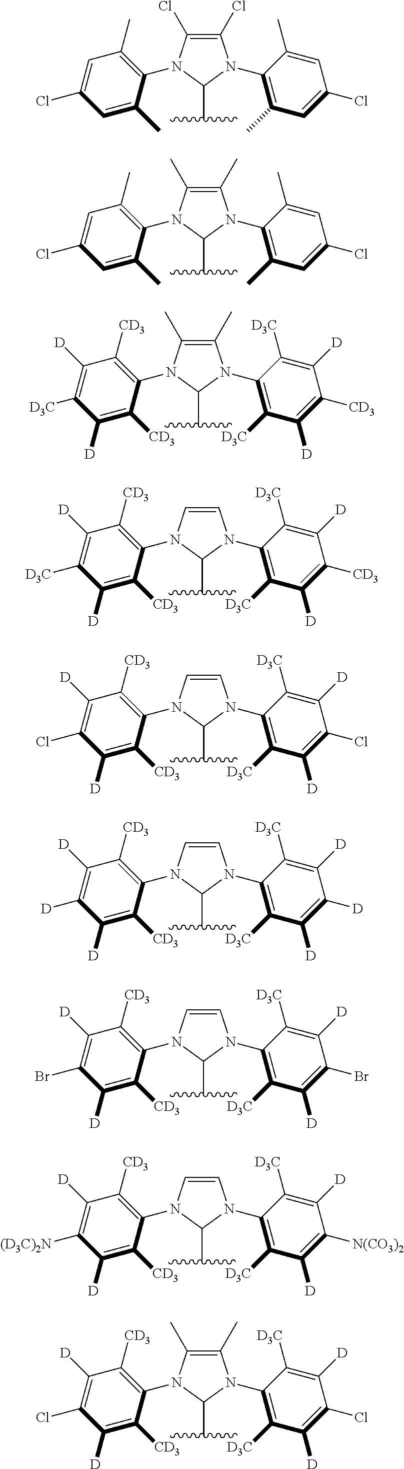

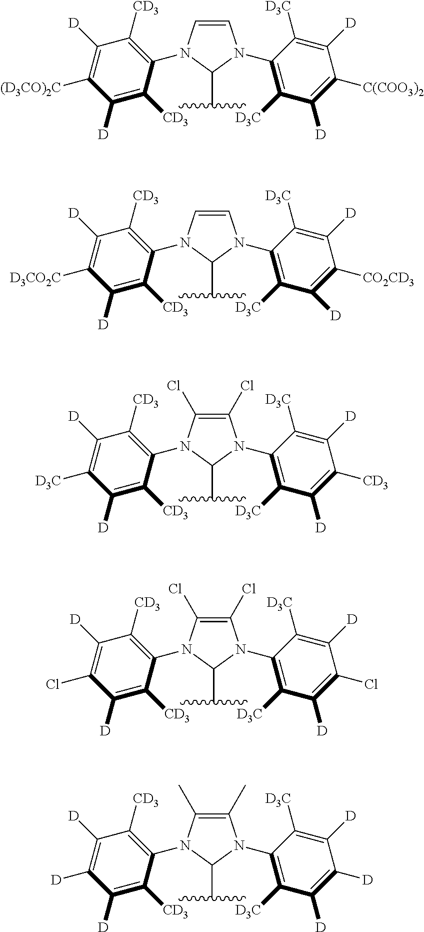

38.-40. (canceled)

41. A method according to claim 1 wherein a biphasic element is introduced into the solvent in order to separate the hyperpolarised target molecule from the transfer catalyst.

42. A method according to claim 41 wherein a biphasic element comprises preparing a fluid containing two separate components, wherein a first solvent is a polar solvent, e.g. DMSO and a second solvent is an immiscible co-solvent e.g. a non-polar solvent, such as, toluene, chloroform or dichloromethane.

43. A method according to claim 41 wherein the ratio of solvent phases is selected to:
(i) maximise the degree of target hyperpolarisation; and/or
(ii) maximise the speed of phase separation.

44. A method according to claim 41 wherein the solvent mixture combination is used to maximise the relaxation time of the hyperpolarised target molecule in the solution by:
(i) employing D₂O;
(ii) employing a D₂O/H₂O mixture of suitable proportion e.g. 1:1; and/or
(iii) adding a further co-solvent to an appropriate aqueous phase such as ethanol or d₆-ethanol.

45. A method according to claim 41 wherein a solvent phase-separation promoter e.g. NaCl or NaO₂CCH₃ or NaOH or NaHCO₃ or Na₂CO₃ or ethanol, at a suitable concentration is added to the system.

46. A method according to claim 45 wherein the concentration of the phase-separation promoter is an amount suitable to:
(i) achieve physiological conditions;
(ii) vary the solutions pH to achieve optimal SABRE;
(iii) optimise organic phase extraction; and/or
(iv) optimise the speed of phase-separation.

47. A method according to claim 45 wherein the phase-separation promoter is suitable for in vivo use and suitable to achieve physiological conditions.

48. A method according to claim 45 wherein the phase-separation promoter is suitable to withstand variations in pH which may be desirable to achieve optimal SABRE.

49.-66. (canceled)
